(19) United States
(12) Patent Application Publication
(10) Pub. No.: US 2026/0066123 A1
(43) Pub. Date: Mar. 5, 2026

(54) Method and Apparatus for a Fully Open AI Foundation Model for Medical Image Analysis

(71) Applicants: DongAo MA, Tempe, AZ (US); Jiaxuan PANG, Tempe, AZ (US); Jianming LIANG, Scottsdale, AZ (US)

(72) Inventors: DongAo MA, Tempe, AZ (US); Jiaxuan PANG, Tempe, AZ (US); Jianming LIANG, Scottsdale, AZ (US)

(73) Assignee: Arizona Regents on behalf of Arizona State University, Scottsdale, AZ (US)

(21) Appl. No.: 19/319,596

(22) Filed: Sep. 4, 2025

Related U.S. Application Data
(60) Provisional application No. 63/690,726, filed on Sep. 4, 2024.

(51) Int. Cl. G16H 50/20; G16H 30/40

(52) U.S. Cl. CPC G16H 50/20; G16H 30/40

(57) ABSTRACT

A pretraining framework for an AI model to learn visual representations from large-scale aggregated medical images by accruing and reusing expert knowledge embedded in all available heterogeneous labels, the framework comprising a teacher model and a student model. The teacher model and the student model are each augmented with multi-task heads, wherein each multi-task head corresponds to one task. The teacher model and student model are each trained via an iterative cyclic pretraining process, in which, at each iteration, the student model is to accrue knowledge from every expert annotation through its corresponding task head by sequentially scanning all tasks one by one for one epoch and, at the end of each task, the knowledge accrued by the student model is accumulated into the teacher model via exponential moving averages (EMA) and reused to help the student model accrue more knowledge from the expert annotations associated with a next task.

### CLAIM OF PRIORITY

This application claims the benefit of U.S. Provisional Patent Application No. 63/690,726, filed Sep. 4, 2024, entitled “AN OPEN FOUNDATION MODELS FOR CHEST RADIOGRAPHY”, the disclosure of which is incorporated by reference herein in its entirety.

### GOVERNMENT RIGHTS AND GOVERNMENT AGENCY SUPPORT NOTICE

This disclosure was made with government support under R01 HL128785 awarded by the National Institutes of Health. The government has certain rights in the disclosure.

### COPYRIGHT NOTICE

This document contains material which is subject to copyright protection. The copyright owner has no objection to the facsimile reproduction by anyone of the document as it appears in the Patent and Trademark Office records but otherwise reserves all copyright rights whatsoever.

### TECHNICAL FIELD

This disclosure relates to an artificial intelligence foundation model for analyzing medical images, such as chest X-rays to diagnose disease, in particular, a pretraining framework for an AI model to learn visual representations from large-scale aggregated medical images by accruing and reusing the expert knowledge embedded in all available heterogeneous labels.

### BACKGROUND

Chest radiography (chest X-ray, CXR) is the most frequently performed radiological exam. Rapid and accurate CXR interpretation is essential for patient care. Artificial intelligence (AI) and deep learning hold transformational power, unlocking the potential to automate CXR interpretation and advance the future of medical diagnostics. However, early deep-learning models were limited in accuracy mainly due to data paucity and biased data sources, restricting their diagnostic scope, generalizability, adaptability, robustness, and extensibility.

Foundation models, trained on large-scale datasets, have emerged as a transformative approach in deep learning. They offer unparalleled capabilities in various applications, including diagnostic imaging. These models, powered by extensive and diverse training data, have matched or surpassed the accuracy of experts in diagnosing abnormalities and have also demonstrated generalizability to clinical situations beyond the scope of their initial training. Such models can democratize access to expert-level diagnostic capabilities, particularly in underserved or remote areas where radiological expertise is scarce.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1A is a functional block diagram of the disclosed embodiments, referred to herein as Ark+, an open foundation model for chest radiography.

FIG. 1B is a functional block diagram of Ark+'s three pretrained components, according to the disclosed embodiments.

FIG. 2A illustrates performance on diagnosing common thoracic diseases, through comparison of six large-scale pretrained models in diagnosing 14 thoracic diseases, showing Ark+ is the top performer in both setups, underscoring its strength as an open foundation model.

FIGS. 2B and 2C illustrate Ark+'s capability for expanding the diagnostic capability and correcting possible overdiagnosis by experts.

FIG. 3A provides an evaluation of adaptability of the disclosed embodiments to evolving diagnostic tasks via linear-probing on the six global labels and 22 local labels from VinDr-CXR.

FIGS. 3B and 3C illustrate the performance for individual global and local labels indicating Ark+ significantly outperforms CXR-FM on all six global labels and 13 local labels according to the disclosed embodiments.

FIG. 3D illustrates Ark+'s adaptability and robustness for detecting rare conditions (Subcutaneous Emphysema, Tortuous Aorta, and Pneumoperitoneum) using 2-way k-shot learning (No Finding versus each rare conditions), according to the disclosed embodiments.

FIGS. 4A and 4B illustrate performance of the disclosed embodiments on handling long-tailed thoracic diseases.

FIG. 4C illustrates the performance of Ark+ and CXR-FM across 19 diseases with varying fractions of training data, according to the disclosed embodiments.

FIGS. 5A-5D illustrate performance of the disclosed embodiments in recognizing common thoracic diseases in new settings without training.

FIG. 6 illustrates the training pipeline of Ark+ according to the disclosed embodiments.

FIG. 7 illustrates a federated learning scenario by distributing Ark+'s training across three local sites, according to the disclosed embodiments.

FIGS. 8A-8G illustrate how the embeddings for COVID-19, Pneumonia and Normal evolve in t-SNE, according to the disclosed embodiments.

FIG. 9 is a functional block diagram of Federated Ark+ according to the disclosed embodiments.

### DETAILED DESCRIPTION

### 1 Introduction

Chest radiography frequently serves as baseline imaging for most lung diseases. Deep learning has great potential for automating chest radiography interpretation. However, existing chest radiographic deep-learning models are limited in diagnostic scope, generalizability, adaptability, robustness, and extensibility. To overcome these limitations, the disclosed embodiments provide Ark+, a foundation model applied to chest radiography, pretrained by cyclically Accruing and Reusing the Knowledge from heterogeneous expert labels with numerous datasets. Ark+ excels in diagnosing thoracic diseases, expanding diagnostic scope, addressing potential over-diagnosis, adapting to rare conditions and new diagnostic settings, tolerating data biases and long-tailed distributions, and supporting federated learning to preserve privacy. Ark+ serves as a model foundational to medical imaging. Ark+'s exceptional capabilities stem from the insight: aggregating various datasets diversifies patient populations and accrues knowledge from many experts, yielding unprecedented performance while reducing annotation costs. Ark+ further reveals open models trained by accruing and reusing knowledge from heterogeneous expert annotations with a multitude of public (big or small) datasets can surpass the performance of proprietary models trained on large data.

TABLE 1
Comparative overview of Ark+ with nine large-scale pretrained models

| | | Model | Parameter | Input | | Training | Data | Full |
|---|---|---|---|---|---|---|---|---|
| Model | Learning type | backbone | size | resolution | Infrastructure | data size | accessibility | Openness |
| Ark⁺ | Supervised | Swin-Large | 197M | 768 × 768 | A100 GPU | 704K | public | Yes |
| CXR-FM¹⁰ | Supervised | EfficientNet-L2 | 480M | 1024 × 1024 | TPUv3 | 821K | public + | No |
| | | | | | | | private | |
| RAD-DINO²⁰ | Self-supervised | ViT-Base | 86M | 518 × 518 | A100 GPU | 882K | public | No |
| MIM-CXR²¹ | Self-supervised | Swin-Base | 88M | 224 × 224 | V100 GPU | 926K | public | No |
| CheSS²² | Self-supervised | ResNet-50 | 25.6M | 512 × 512 | V100 GPU | 4.8M | private | No |
| KAD⁹ | Image-text | ResNet-50 | 25.6M | 512 × 512 | Not | 377K | public | Yes |
| | | | | | reported | | | |
| ELIXR¹² | Image-text | ELIXR-C + | Not | 1280 × 1280 | TPUv3 | 893K | public + | No |
| | | Q-Former | reported | | | | private | |
| XRV-ResNet³⁰ | Supervised | ResNet-50 | 25.6M | 512 × 512 | Not | 201K | public | Yes |
| XRV-DenseNet³⁰ | | DenseNet-121 | 7.98M | 224 × 224 | reported | | | |

Not all proprietary high-performance foundation models are fully open, as seen in Table 1 above. Ark+, KAD, XRV are considered “fully open”: open source, open model, open weights, and open code and data for pretraining and evaluation. By contrast, no pretraining code was released for CXR-FM, RAD-DINO, MIM-CXR, and ELIXR; no pretraining data were released for CheSS.

This restriction makes it difficult for researchers and developers to build upon existing works, hindering the pace of innovation. The disclosed embodiments envision a fully open, powerful, and robust foundation model that can be trained by aggregating numerous (large or small) public datasets with options of federating private data, thereby fully accessible to the public. Open foundation models enable continuous improvement and adaptation. With public access and openness to contribution, researchers can iteratively refine and enhance these models, ensuring the models remain up to date with the latest medical knowledge and advancements. This ongoing development cycle can lead to more accurate and reliable diagnostic tools, advancing the field of AI-driven healthcare, and ultimately improving patient outcomes.

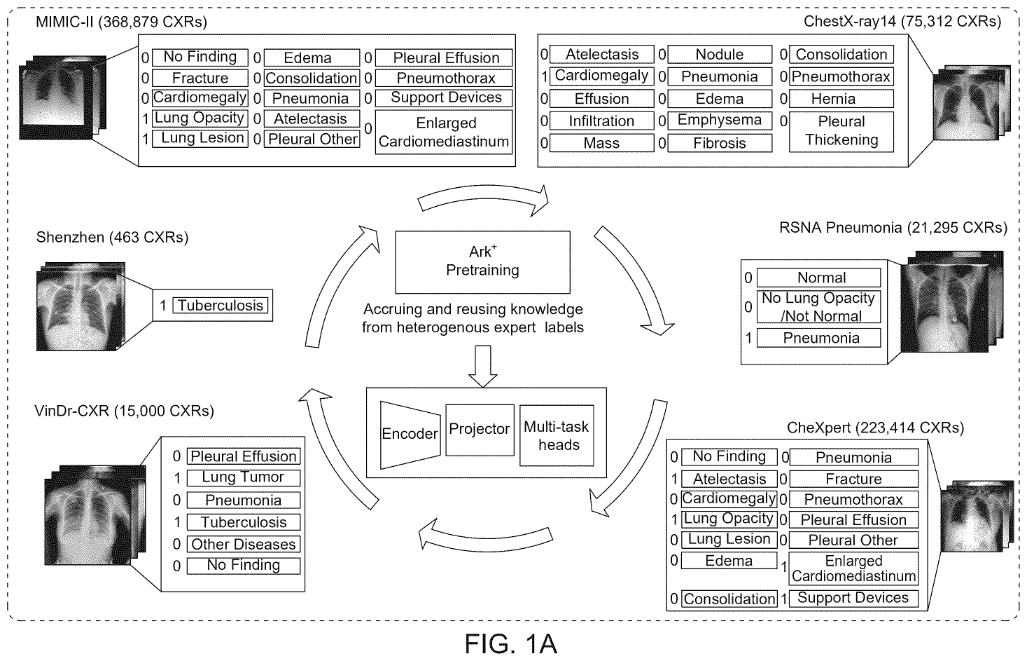

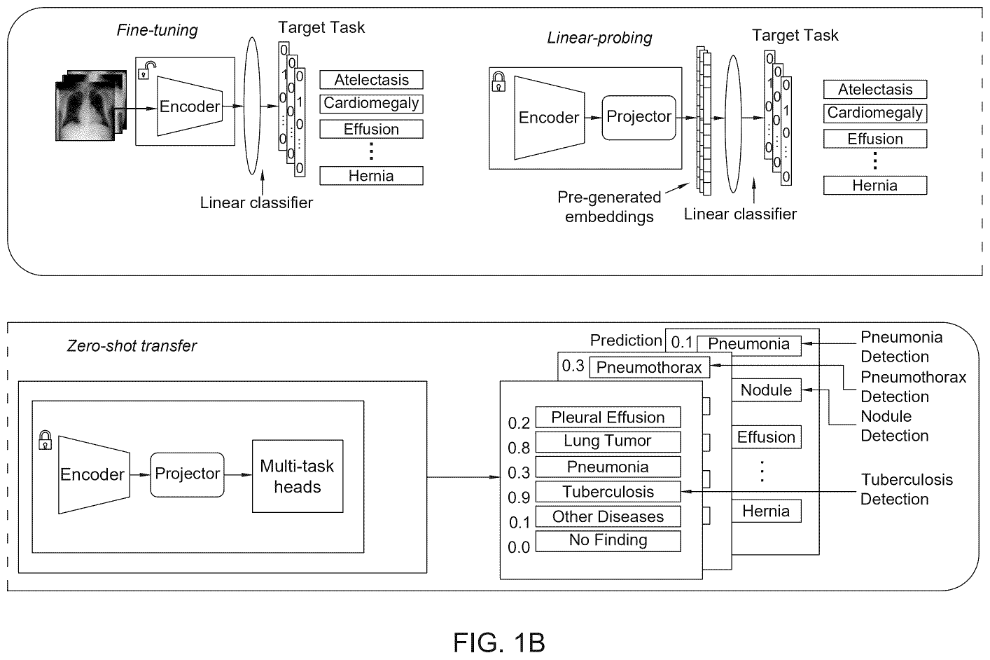

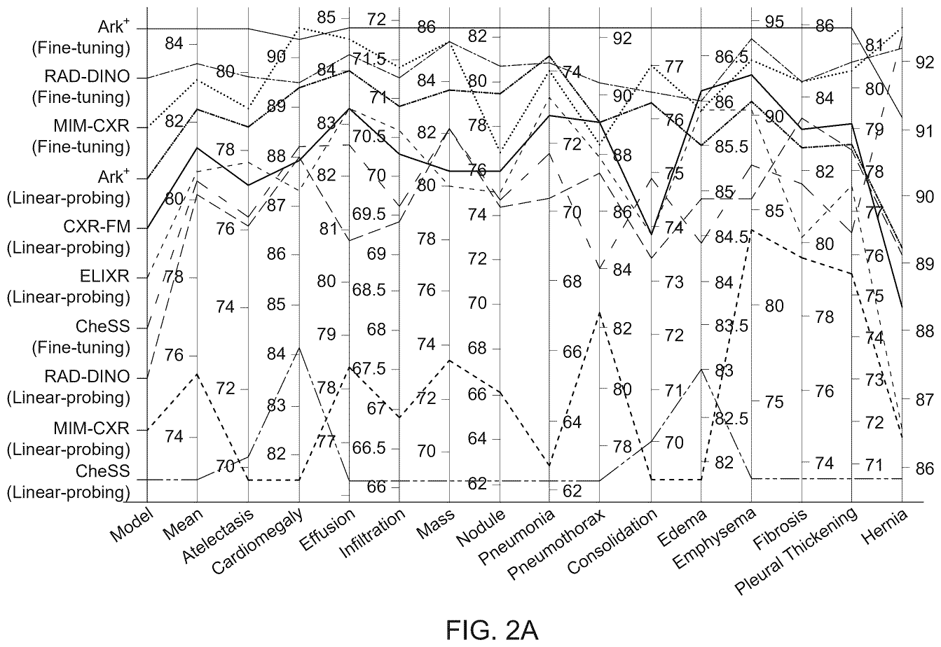

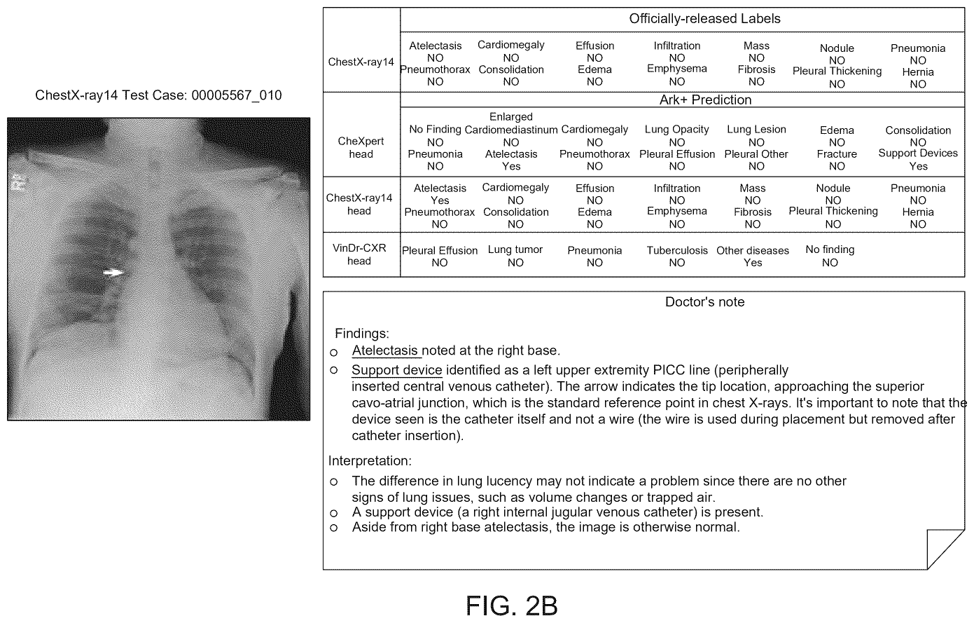

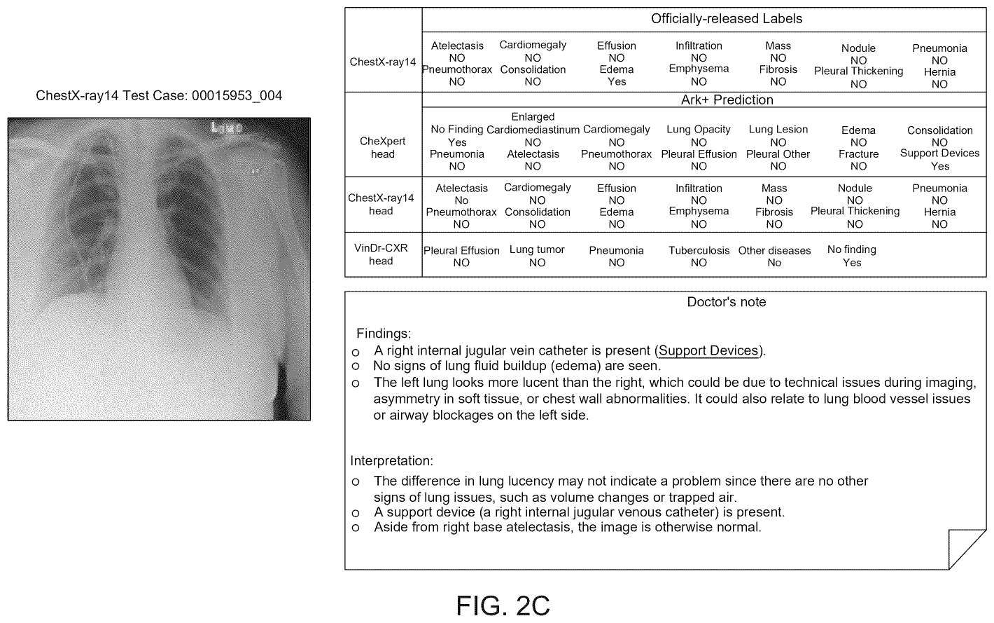

The disclosed embodiments provide a fully open foundation model for medical image analysis, for example chest radiography analysis, referred to herein as Ark+, which is pretrained by cyclically Accruing and Reusing the Knowledge embedded in the heterogeneous expert labels from six public datasets. See the functional block diagram in FIG. 1A of Ark+, pretrained in a cyclical manner by accruing and reusing the knowledge embedded in the heterogeneous expert labels from six public datasets: ChestX-ray14, RSNA Pneumonia, CheXpert, VinDr-CXR, Shenzhen CXR, and MIMIC-II (detailed in FIGS. 1A, 1B, and Table 7), according to the disclosed embodiments. Ark+ builds upon prior work, Ark, offering several enhancements: a larger backbone model using Swin Transformer Large (Swin-Large), an increased image input resolution of 768×768, and a rearranged data augmentation approach that feeds the teacher model the resized original image rather than using random cropping. Ark+ has three pretrained components: an encoder, a projector, and multi-task heads, all of which are openly available for use in various clinical (target) tasks: via fine-tuning, linear-probing, or zero-shot transfer. See FIG. 1B in which Ark+ has three pretrained components: an encoder, a projector, and multi-task heads, which can be adopted to accomplish various clinical (target) tasks via fine-tuning, linear-probing, or zero-shot transfer. To fine-tune Ark+ for a target task, a new randomly-initialized linear classifier was attached to the pretrained encoder and the entire model trained using the images and their associated labels provided by the target task. For linear-probing, the pretrained encoder and projector were first used to generate the embeddings (information-rich numerical vectors) for all images in the target task and then a new linear classifier trained. For zero-shot transfer, the entire pretrained Ark+, including the encoder, projector, and multi-task heads, was used to acquire the prediction directly for each image in the target task, requiring no further training. This disclosure reports internal evaluations on four datasets and external evaluations on six unseen datasets (Table 2) to assess Ark+'s capability relative to nine large-scale pretrained models (Table 1) in diagnosing thoracic diseases across seven different scenarios with the performance included in FIGS. 2A to 5D and Tables 4 and 5. Fine-tuning involves retraining the entire network, including the backbone encoder and the linear classifier. This process harnesses the model's full discriminative power, enabling it to comprehensively adapt to the target task. Linear-probing, on the other hand, focuses on training only a linear classifier atop the frozen features extracted by the pretrained backbone. This approach efficiently applies the model's learned knowledge to the target task, effectively testing the quality of the model's features. Zero-shot transfer directly utilizes the model to diagnose conditions for datasets it has not seen during its pretraining phase. This method tests the model's capacity to generalize its learned knowledge to new datasets without the need for further training or exposure to specific samples from these datasets.

TABLE 2
Overview of datasets utilized for evaluating Ark+

| Dataset | Clinical conditions | Configuration | #Train | #Test |
|---|---|---|---|---|
| Internal | | | | |
| ChestX-ray14¹⁹ | 14 thoracic diseases | fine-tuning, | 75,312 | 25,696 |
| | | linear-probing | | |
| VinDr-CXR⁴² | 6 image-level and 22 lesion- | linear-probing | 15,000 | 3,000 |
| | level thoracic findings | | | |
| CXR-LT*²⁶ | Subcutaneous Emphysema | k-shot learning | 2-10 | 496-488 |
| (rare condition) | Tortuous Aorta | (linear-probing) | 2-10 | 477-469 |
| | Pneumoperitoneum | | 2-10 | 428-420 |
| CheXpert³⁴ | 14 thoracic findings | sex bias study | following | |
| | | (linear-probing) | Larrazabal et al.³³ | |
| External | | | | |
| ChestDR²⁶ | 19 thoracic diseases | fine-tuning, | 979 | 3,869 |
| | | linear-probing | | |
| SIIM-ACR^{†46} | Pneumothorax | zero-shot transfer | 0 | 12,046 |
| NODE21^{‡49} | Nodule | zero-shot transfer | 0 | 4,265 |
| TBX-11K⁴⁷ | Tuberculosis | zero-shot transfer | 0 | 8,400 |
| Mendeley-V2⁴⁸ | Pediatric pneumonia | zero-shot transfer | 0 | 5,856 |
| COVIDxCXR-3³⁵ | COVID-19 and Pneumonia | fine-tuning, | 29,634 | 400 |
| | | linear-probing | | |

As illustrated in Table 2, this disclosure evaluates Ark+ using 10 datasets-four seen datasets for an internal evaluation and six unseen datasets for an external evaluation—by following the official training/testing data splits provided by each dataset, except for CXR-LT (rare disease) in few-shot learning, where 1 to 5 samples with the disease are randomly sampled and an equal number of samples are labeled No Finding for training, using all remaining samples for testing. CXR-LT expands upon MIMIC-II with 12 new classes, among which three rare diseases are selected for our few-shot learning. To ensure no samples are seen by Ark+ during its pretraining, only images from the hold-out validation and test sets of MIMIC-II were utilized. SIIM-ACR, originally for pneumothorax segmentation, is converted into a classification task for zero-shot pneumothorax detection. 617 images shared with the ChestX-ray14 dataset have been excluded to ensure an external evaluation for Ark+.

To assess Ark+'s performance in diagnosing thoracic diseases, internal evaluations were conducted on four datasets and external evaluations were conducted on six datasets (Table 2). Internal evaluations focus on assessing the model using data similar to its training domain, specifically using hold-out test data from a “seen” dataset involved in the model's pretraining. These evaluations provide insights into the model's effectiveness within familiar contexts. External evaluations involve assessing the model using “unseen” datasets from different sites or hospitals that serve different populations and employ different imaging protocols. This type of evaluation is crucial for understanding how well the model can perform in real-world scenarios that may significantly differ from its training environment. Using these 10 different datasets, Ark+ was evaluated across eight different scenarios: (1) diagnosing common thoracic diseases, (2) adapting to evolving diagnostic needs, (3) learning to diagnose rare conditions from a few samples, (4) handling long-tailed thoracic diseases, (5) adjusting to diagnostic setting shifts without training, (6) tolerating the sex-related bias, (7) responding to novel thoracic diseases, and (8) utilizing private data while preserving patient privacy with federated pretraining.

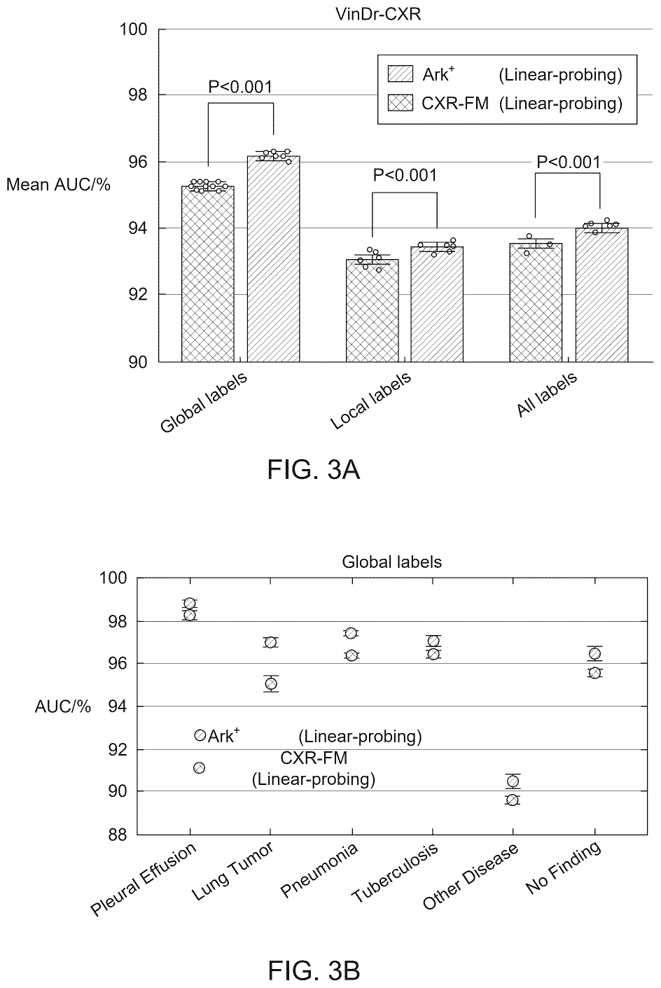

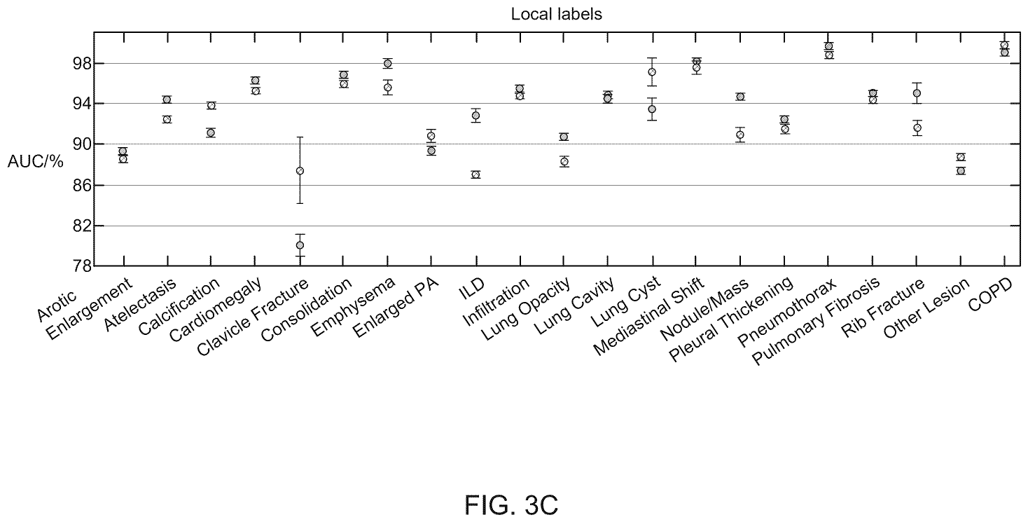

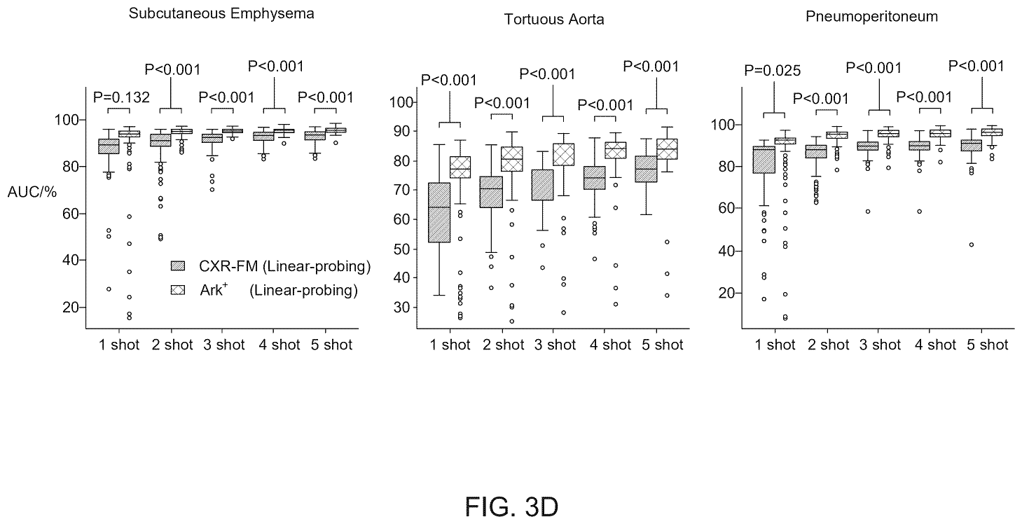

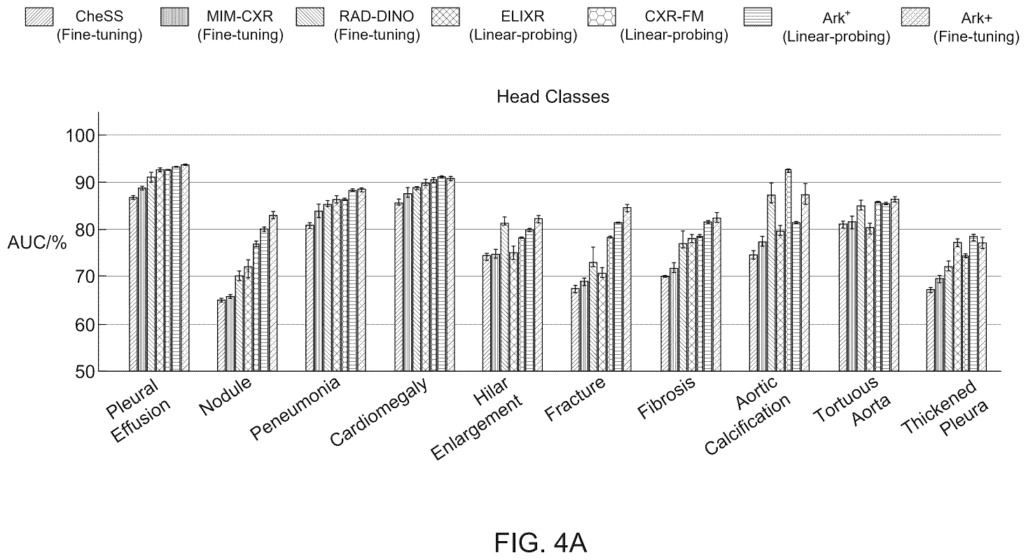

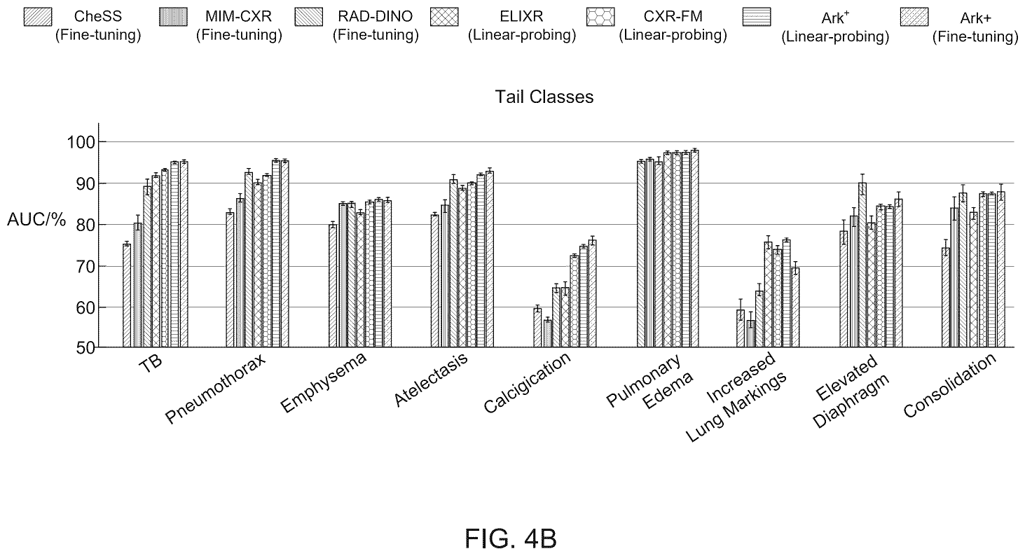

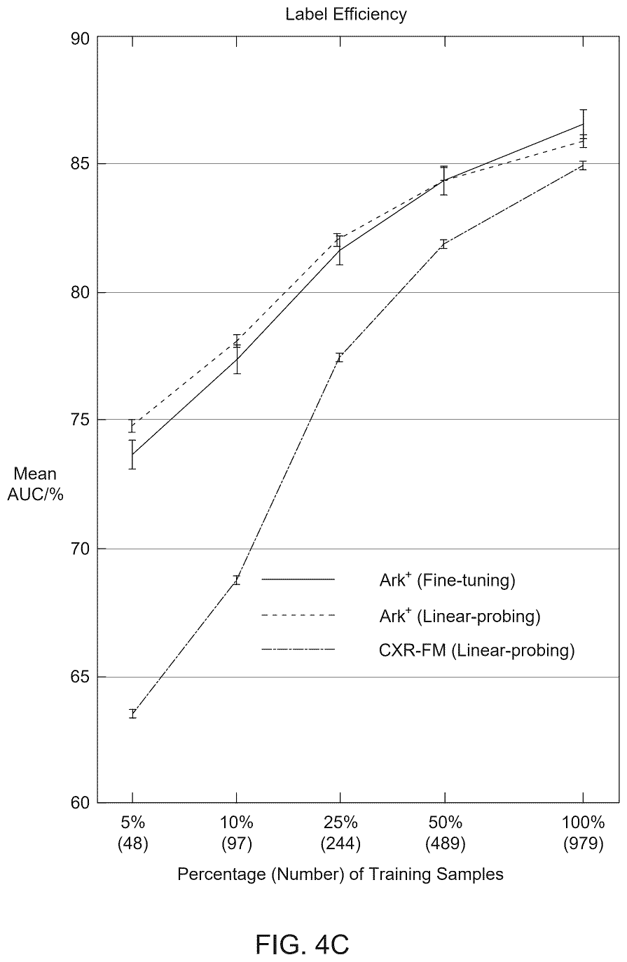

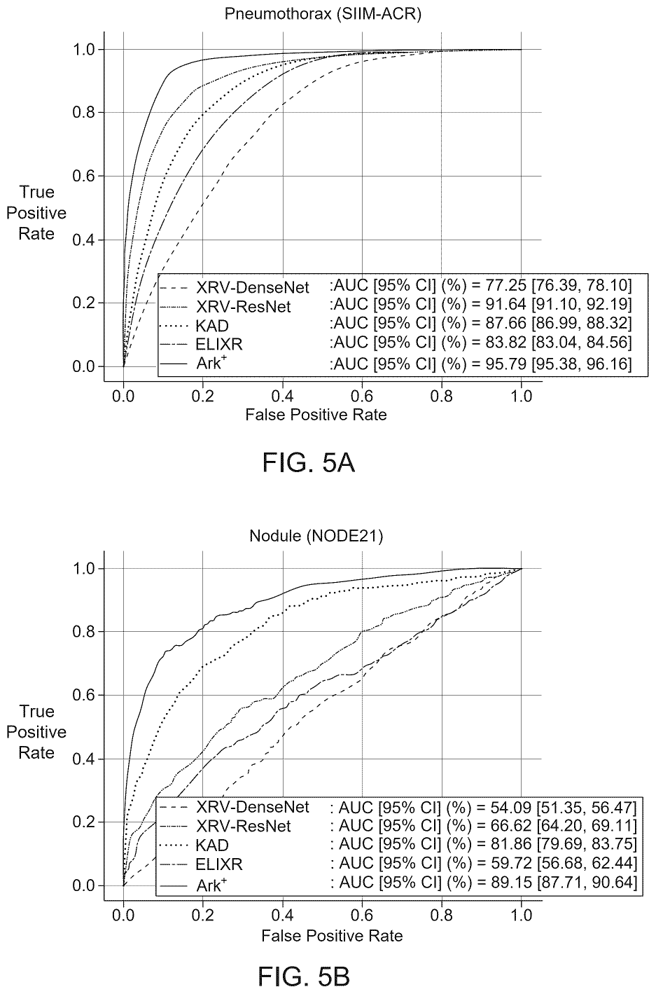

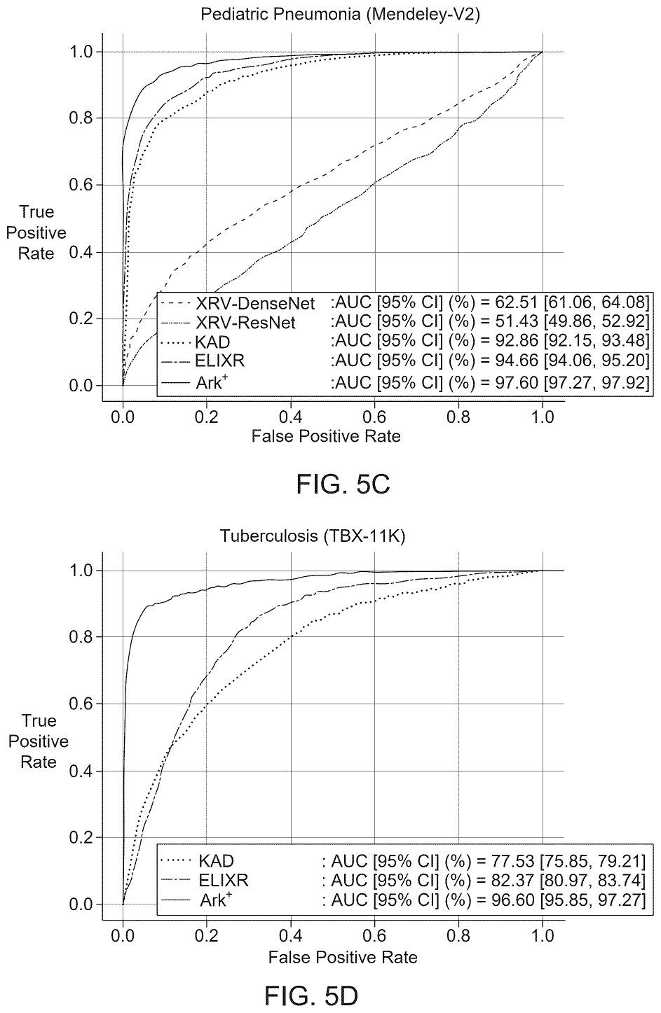

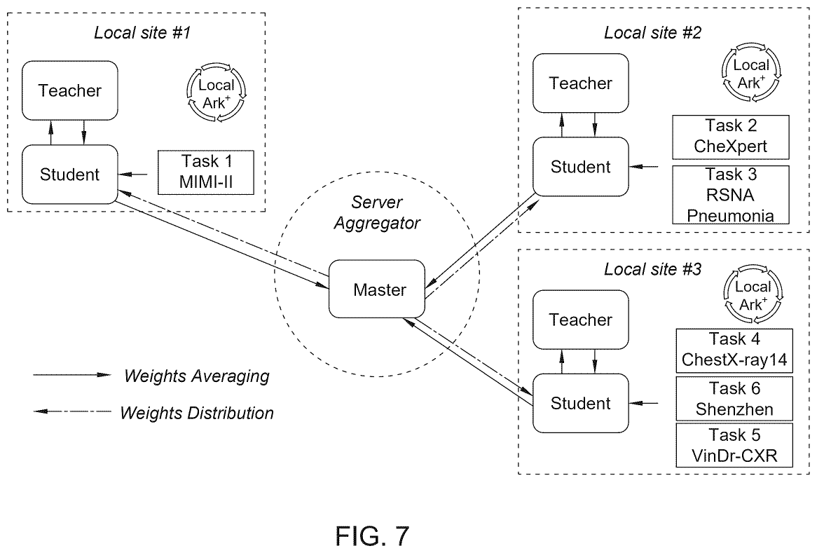

The experimental results demonstrate Ark+'s superior generalizability, adaptability, robustness, and extensibility compared with other foundation models (Table 1) across various clinical scenarios. Generalizability (quantitatively assessed in FIGS. 2A-2C) refers to the model's ability to perform effectively on new data, indicating how the learned knowledge from the training data can be applied across different but related data or clinical scenarios. Adaptability extends generalizability by emphasizing the model's capacity to adjust to and excel in diverse conditions such as new datasets, tasks, or clinical settings, as demonstrated in FIGS. 3A-3C and 5A-5D. Robustness, highlighted in FIGS. 4A-4B and Table 4, reflects the model's ability to maintain high performance despite challenges such as long-tailed data distributions or biased training data. Extensibility is the ability to incorporate novel tasks and scenarios, as evidenced in Ark+'s integration of a (new) COVID-19 diagnostic task via incremental learning (Table 5) and extension to federated learning for incorporating private data, preserving patient privacy, and distributing pretraining (FIG. 7 and Table 6), in addition to its augmentation beyond classification to support localization, segmentation, and their integration. To comprehensively evaluate performance, metrics were adopted, including Area Under the ROC Curve (AUC), Accuracy (ACC), F1 Score, Average Precision (AP), and Matthews Correlation Coefficient (MCC). Statistical significance between models was assessed using p-values from the two-sided independent t-test, and 95% confidence intervals were calculated for zero-shot transfer results based on 1,000 resampled test sets.

### 2 Results

### 2.1 Diagnose Common Thoracic Diseases

An internal evaluation of Ark+ was conducted using the hold-out test data from ChestX-ray14, a well-established public dataset commonly used for benchmarking model performance in diagnosing 14 common thoracic diseases (FIGS. 2A-2C), under both fine-tuning and linear-probing setups, relative to the large-scale pretrained models: CXR-FM, ELIXR, RAD-DINO, MIM-CXR, and CheSS. As illustrated in FIG. 2A, Ark+ achieves the highest average performance in both setups, showcasing its generalizability. The fine-tuning results, depicted in FIG. 2A, show that Ark+ achieves a mean AUC of 84.43±0.09% over the 14 diseases, surpassing RAD-DINO, MIM-CXR and CheSS by a significant margin of 0.89%, 1.35% and 3.97%, respectively. In the linear-probing results, Ark+ consistently ranks at the top, while CXR-FM ranks second. Both models significantly outperform the self-supervised models-RAD-DINO, MIM-CXR, and CheSS—by a considerable margin, showing the power of learning from expert labels. Moreover, fine-tuning consistently surpasses linear-probing for all models, underscoring the importance of foundation model openness for effective local adaptation through fine-tuning.

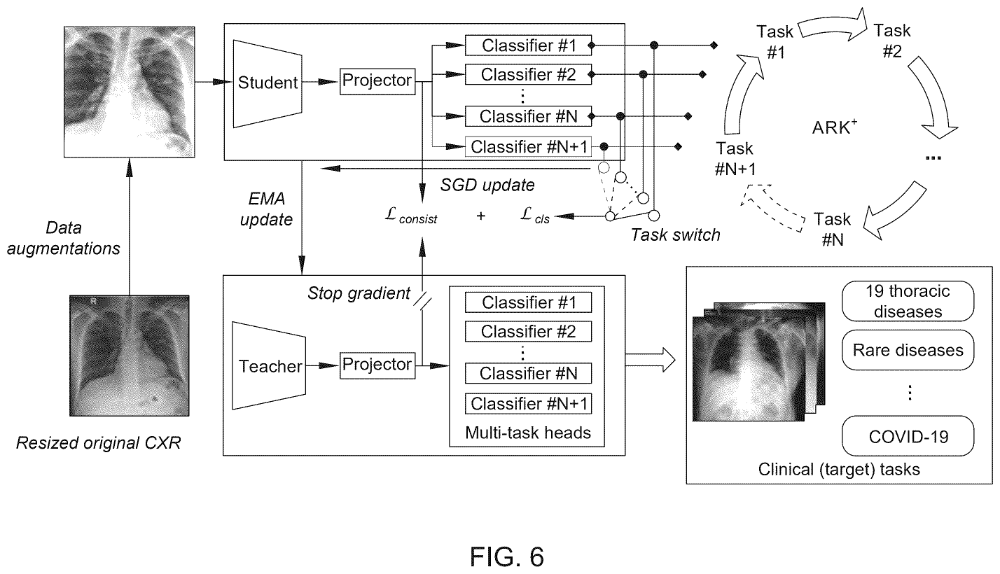

Additionally, Ark+ shows the capability to expand the diagnostic scope and correct possible overdiagnosis by experts using its multi-task heads (see FIG. 6) and by pretraining on diverse tasks (See FIG. 1A-1B). FIG. 6 illustrates how Ark+ is built on a teacher-student framework augmented with multi-task heads, each corresponding to a specific task, and employs cyclic pretraining to iteratively accrue and reuse knowledge. At each iteration, the student model sequentially scans datasets (tasks) one by one for one epoch, learning from expert annotations through the task-specific head. The knowledge accrued by the student is accumulated into the teacher via exponential moving averages (EMA), enabling the teacher to guide the student in subsequent tasks. To reinforce the feedback loop between the student and teacher, after their encoders, a projector is introduced to map the representations to the same feature space via the consistency loss, also serving as the embedding for linear-probing in our evaluation. After pretraining, the accumulated knowledge in the teacher can be reused and transferred to target tasks. Differing from the previous design in Ark, Ark+ feeds the teacher with the resized original image instead of random cropping. This update in data augmentation ensures the teacher provides a consistent and steady supervisory signal for computing the consistency loss, thereby accelerating training and enhancing performance. The multi-task heads generate diagnostic results from both their specific task and other heads pretrained on different tasks, enabling a broader diagnostic scope and more comprehensive, accurate diagnoses while effectively managing overdiagnosis. To illustrate this capability, two cases are presented in FIGS. 2B and 2C, with the officially-released labels from ChestX-ray14, diagnostic results from Ark+, and the doctor's note shown below. The 14 official disease labels for the images are derived using natural language processing from the associated radiological reports written by human experts. Ark+'s diagnostic results are obtained from the CheXpert head, the ChestX-ray14 head, and the VinDr-CXR head, with thresholds selected using validation data. Cases where Ark+ disagrees with the official labels were reviewed by a senior cardiopulmonary Mayo Clinic radiologist with 30 years of experience for verification. In FIG. 2B, the official label indicates no findings for the case, while Ark+ predicts Atelectasis and Support Devices. The doctor's note confirms Ark+'s diagnosis as correct, demonstrating that Ark+ not only detects a previously undiagnosed condition but also expands diagnostic capabilities to identify Support Devices. In FIG. 2C, the official label indicates Edema, but Ark+ finds no evidence of Edema and instead confirms No Finding based on the CheXpert and VinDr-CXR heads. This diagnosis aligns with the doctor's opinion, demonstrating Ark+'s ability to correct possible overdiagnosis by ChestX-ray14 experts. Moreover, Ark+'s prediction of Support Devices further underscores its expanded diagnostic capabilities.

### 2.2 Adapt to Evolving Diagnostic Needs

Annotating medical images is tedious, labor-intensive, and time-consuming, requiring costly specialty-oriented skills that are not easily accessible. Consequently, medical datasets are often partially labeled, incompletely addressing clinical needs, resulting in incompletely trained models with comparatively restricted adaptability. Furthermore, evolving medical practices and clinical applications necessitate updates to datasets (e.g., recognition of new conditions) and upgrades or complete retraining of AI models to meet these changing needs. To demonstrate Ark+'s adaptability to evolving diagnostic needs, when pretraining Ark+, only six image-level global labels were included from VinDr-CXR, and its 22 lesion-level local labels were excluded, meaning that Ark+ has never been trained with the 22 local diseases. Specifically, during pretraining, even though Ark+ “saw” all chest radiographs in the VinDr-CXR training set, including those with any of the 22 local diseases, it did not use these specific disease labels to guide its learning process. As a result, chest radiographs with none of the five global diseases were treated, intentionally albeit incorrectly, as “purely normal” (i.e, No Finding) when Ark+ was pretrained, allowing assessment of Ark+'s adaptability to those 22 local diseases after the pretraining.

Both Ark+ and CXR-FM were evaluated on their adaptabilities via linear-probing, reporting the average AUC scores and the standard deviations (%) across 10 runs for each disease on the VinDr-CXR hold-out test set. As shown in FIG. 3A, Ark+ achieves mean AUC scores of 96.19±0.09%, 93.45±0.10%, and 94.06±0.09% for the six global labels, the 21 local labels, and all 27 labels, respectively, significantly outperforming CXR-FM, which achieves 95.29±0.07%, 93.06±0.16%, and 93.55±0.12%, respectively. Note that while VinDr-CXR is external to CXR-FM, it performs strongly on this task. For global labels (FIG. 3B), Ark+ achieves an AUC above 90% for all findings, surpassing CXR-FM in every case. Among the 21 local labels (FIG. 3C), Ark+ achieves an AUC above 90% on 17 findings and outperforms CXR-FM on 14 findings. In particular, Ark+ excels on the common thoracic diseases encountered during pretraining, such as Atelectasis, Cardiomegaly, Consolidation, Emphysema, Infiltration, Nodule/Mass, Pneumothorax, and Fibrosis. These results highlight Ark+'s ability to adapt to evolving clinical needs without requiring extensive retraining, making it a valuable tool in real-world applications where adaptability and accuracy are essential. Note that while VinDr-CXR's official training set includes samples for all 22 local labels, its test set contains no samples labeled Edema; therefore, no performance is reported in FIG. 3C for Edema.

2.3 Learn Rare Conditions from a Few Samples

Timely and accurate diagnosis of rare diseases is challenging yet crucial for improving patient outcomes and optimizing treatment strategies. Due to their low prevalence, the available training samples for rare diseases are very limited, restricting the model's diagnostic accuracy. Therefore, a critical measure of a foundation model's effectiveness and practical utility in clinical settings is its ability to adapt to rare disease detection using only a few labeled samples via few-shot learning. For this purpose, 2-way (No Finding versus a condition present) k-shot learning was conducted to assess Ark+'s few-shot learning ability, comparing its performance with CXR-FM. Both models were evaluated under the linear-probing configuration. For this study, three rare conditions were selected: Subcutaneous Emphysema, Tortuous Aorta and Pneumoperitoneum, and a benchmark was established, built upon MIMIC-II using the labels from the CXR-LT challenge. To ensure Ark+ had no prior exposure to these samples during pretraining, the relevant images were removed from the training set, yielding 78 samples for Subcutaneous Emphysema, 59 samples for Tortuous Aorta, and 10 samples for Pneumoperitoneum. Each of these three sets was combined with 420 samples labeled No Finding to create three rare condition datasets. For each trial, k samples were randomly selected with the condition and k samples labeled No Finding to train a linear classifier, evaluating performance on the remaining samples.

The box plot in FIG. 3D depicts the distribution of AUC scores for Ark+ and CXR-FM across 100 experimental replicas. Ark+ consistently outperforms CXR-FM, achieving higher median and maximum AUC scores with a narrower interquartile range in all experiments, underscoring its superior performance. However, both models exhibit outliers with AUC scores below 50%, reflecting the inherent difficulty of detecting rare conditions with very limited samples. These findings demonstrate Ark+'s capabilities to effectively diagnose rare disorders using only a few labeled samples, highlighting its potential for practical clinical applications in scenarios with limited data availability.

### 2.4 Handle Long-Tailed Thoracic Diseases

Chest radiographic diagnosis often presents a long-tailed problem—the distribution of clinical findings is skewed towards a few common abnormalities while rare conditions are less frequently encountered, posing challenges for training models due to the imbalance in class frequencies. Models often prioritize learning from the majority classes, leading to biases toward common findings and therefore potentially neglecting rare conditions. Moreover, the scarcity of data for rare conditions can result in overfitting to the limited samples, reducing the model's ability to generalize effectively to unseen cases. Therefore, robustness and generalizability to long-tailed scenarios are key indicators for evaluating foundation models.

To assess Ark+'s performance in long-tailed scenarios, the unseen dataset ChestDR was used, which was developed for examining the large-scale foundation models in diagnosing 19 thoracic diseases with long-tailed distributions. Ark+ was fine-tuned using all training data and 19 disease labels, and its performance was compared with the fine-tuning results of RAD-DINO, MIM-CXR, and CheSS, as well as the linear-probing results of CXR-FM and ELIXR. The results, plotted in FIGS. 4A-4B, show that Ark+ outperforms other models across 16 of the 19 diseases. Consistent with the findings in FIG. 2A, supervised models Ark+ and CXR-FM demonstrate a significant advantage over self-supervised models RAD-DINO, MIM-CXR, and CheSS as well as image-text model ELIXR built on CXR-FM. For instance, Ark+ achieves a mean AUC of 86.55±0.35% across the 19 diseases through fine-tuning, significantly surpassing RAD-DINO's 82.73±0.57%, MIM-CXR's 78.03±0.95%, and CheSS's 75.86±0.10%. Table 3 offers a more comprehensive evaluation, presenting the mean MCC, mean AP, and mean F1 scores for the 19 classes. Each model was trained using all 979 available samples and evaluated on the 3,869 hold-out test samples. The table reports the mean and standard deviation of four metrics across 10 independent trials.

TABLE 3
Performance on diagnosing 19 thoracic diseases with long-tailed distributions

| Configuration | Model | meanAUC(%) | meanMCC(%) | meanAP(%) | meanF1(%) |
|---|---|---|---|---|---|
| linear-probing | CheSS | 73.20 ± 0.02 | 21.34 ± 0.08 | 24.53 ± 0.06 | 26.91 ± 0.13 |
| | MIM-CXR | 72.14 ± 0.03 | 20.45 ± 0.09 | 23.24 ± 0.05 | 26.11 ± 0.12 |
| | RAD-DINO | 79.18 ± 0.06 | 27.53 ± 0.14 | 33.49 ± 0.07 | 31.15 ± 0.16 |
| | ELIXR | 81.97 ± 0.24 | 29.95 ± 0.46 | 34.28 ± 0.47 | 32.60 ± 0.47 |
| | CXR-FM | 84.93 ± 0.10 | 33.29 ± 0.29 | 40.02 ± 0.15 | 32.68 ± 1.26 |
| | Ark⁺ | 85.92 ± 0.06 | 35.70 ± 0.21 | 42.96 ± 0.10 | 34.75 ± 0.43 |
| fine-tuning | CheSS | 75.86 ± 0.10 | 23.31 ± 0.29 | 25.15 ± 0.26 | 27.63 ± 0.31 |
| | MIM-CXR | 78.03 ± 0.95 | 26.26 ± 0.92 | 28.06 ± 1.01 | 28.99 ± 0.58 |
| | RAD-DINO | 82.73 ± 0.57 | 31.10 ± 1.16 | 37.24 ± 1.90 | 33.80 ± 1.11 |
| | Ark⁺ | 86.55 ± 0.35 | 36.90 ± 0.55 | 45.69 ± 0.84 | 38.81 ± 0.55 |

To further examine label efficiency, the training data was reduced to 50%, 25%, 10%, and 5% of the full dataset and Ark+ was evaluated under both fine-tuning and linear-probing setups, comparing its performance with CXR-FM's linear-probing results. As shown in FIG. 4C, Ark+ significantly outperforms CXR-FM in low-data scenarios. For instance, under the same linear-probing setup, Ark+ exceeds CXR-FM's mean AUC by 11.29%, 9.33%, and 4.75% when using 5%, 10%, and 25% of the training samples, respectively. Interestingly, in these limited-data scenarios, Ark+ demonstrates better performance with linear-probing than fine-tuning. However, as training data increases, fine-tuning surpasses linear-probing, further boosting performance. This underscores the significance of an open model, particularly when sufficient training data are available.

2.5 Transfer to New Sites without Training

Domain shifts, caused by variations in patient populations, scanner types, and imaging protocols across hospitals, can hinder deep learning model generalization and accuracy. While transfer learning and domain adaptation techniques can address domain shift issues, they rely on access to data and labels from the target domain for retraining or fine-tuning, limiting the scalability and widespread deployment of foundation models.

To evaluate Ark+'s adaptability in generalizing knowledge acquired during pretraining to unseen domains (data from new sites) without additional training, experiments were conducted on a diverse range of datasets representing various clinical scenarios. The four datasets were collected from distinct sources and focused on different clinical objectives, including the detection of Pneumothorax, Nodule, Pediatric Pneumonia, and Tuberculosis. Ark+ performed zero-shot transfer to detect these diseases using its pretrained multi-task heads, without any task-specific training. For comparison, two supervised models were evaluated from TorchXRay Vision (XRV), trained on multiple datasets, and two image-text pretrained models (i.e., KAD and ELIXR), under the same zero-shot transfer conditions.

The ROC curves with 95% confidence intervals, shown in FIGS. 5A-5D, illustrate the zero-shot transfer performance for disease detection across the datasets. Ark+ achieves impressive AUC scores for detecting Pneumothorax (95.79%), Pediatric Pneumonia (97.60%), and Tuberculosis (96.60%). For Nodule detection, where the images from ChestX-ray14 have been excluded, Ark+ attains an AUC score of 89.15%, significantly outperforming all other models. These results underscore Ark+'s capacity to transfer to unseen domains and diverse clinical contexts without training, highlighting its potential for real-world deployment.

### 2.6 Tolerate Sex-Related Bias

Population imbalance is a prevalent issue in medical datasets, often leading to the development of biased models. These biases result in suboptimal diagnostic performance for minority populations that are underrepresented in the training data, raising ethical concerns about equity and inclusivity in healthcare. A robust foundation model must demonstrate resilience against biased training data, delivering equitable and accurate diagnostics across all population groups.

To evaluate the model's tolerance to sex-related bias, the methodology of Larrazabal et al. was followed under a linear-probing configuration, using sex-exclusive training sets of CheXpert. Larrazabal, A. J., Nieto, N., Peterson, V., Milone, D. H. & Ferrante, E. Gender imbalance in medical imaging datasets produces biased classifiers for computer-aided diagnosis. Proceedings of the National Academy of Sciences 117, 12592-12594 (2020). The evaluation adhered to their train/test splits to ensure a balanced number of cases per class across 20 male-only and 20 female-only folds, where the labels “No Finding” and “Support Device” were excluded. 40 linear classifiers were trained on the male-only and female-only splits using embeddings from each model and these classifiers evaluated on the corresponding male-only and female-only test splits. Bias was assessed by identifying statistically significant performance declines when the training and test data came from opposite sexes compared with when they came from the same sex. For example, when tested on female-only sets, classifiers trained on female-only embeddings should not significantly outperform those trained on male-only embeddings unless bias exists.

Table 4 presents the evaluation results for four large-scale pretrained foundation models, assessing their robustness against sex-related bias. Results without statistically significant performance differences, highlighted in green, indicate robustness when training data and test data differ in sex. Ark+ demonstrates exceptional resilience, with 13 unbiased results, outperforming CheSS's 4, MIM-CXR's 5, and CXR-FM's 8. These findings underscore Ark+'s superior ability to mitigate the effects of imbalanced data and reduce sex-related biases, enhancing equity and accuracy in computer-aided diagnosis.

In Table 4, All models are evaluated on CheXpert via linear-probing using sex-exclusive training/test splits by following Larrazabal et al., Ark+ has 13 unbiased results (bolded), demonstrating the greatest resilience to sex-imbalanced data compared with the other models. Sex bias is characterized by a significant drop in performance when training and test data are of the opposite sex compared to when they are of the same sex. A robust model should have more unbiased results that do not show a statistically significant performance difference (at p=0.05) between datasets exclusively composed of male or female data. All unbiased results are highlighted in gray.

In Table 4, the abbreviations are defined as follows: Dz: Disease, EC: Enlarged Cardiomediastinum, CM: Cardiomegaly, LO: Lung Opacity, LL: Lung Lesion, ED: Edema, CS: Consolidation, PN: Pneumonia, AT: Atelectasis, PX: Pneumothorax, PE: Pleural Effusion, PO: Pleural Other, FR: Fracture. The greater number of unbiased results indicate that the model is more effective in tolerating sex-related biases.

### 2.7 Respond to Novel Diseases

The COVID-19 pandemic, caused by the SARS-COV-2 virus outbreak in 2020, profoundly impacted global health. Effective screening of infected individuals became crucial for timely treatment, care, and virus containment. While RT-PCR testing is a standard diagnostic method, such testing was not available during the initial months of the pandemic, and CXR offers a rapid approach to identifying COVID-19-related lung abnormalities. However, the surge in patient numbers during the pandemic overwhelmed radiologists, highlighting the need for diagnostic models to assist in the efficient and accurate detection of COVID-19 cases. This global health crisis presented a unique opportunity to evaluate a foundation model's ability to adapt to novel diseases and support timely and accurate diagnosis. To this end, Ark+'s adaptability for distinguishing COVID-19, Pneumonia and Normal cases was assessed using the COVID×CXR-3 dataset, and compared with CXR-FM, MIM-CXR, and RAD-DINO. Additionally, to demonstrate Ark+'s extensibility to novel diseases, Ark+ was incrementally and continually pretrained with the COVID×CXR-3 training data, producing an updated foundation model referred to as Ark+_{+covid}.

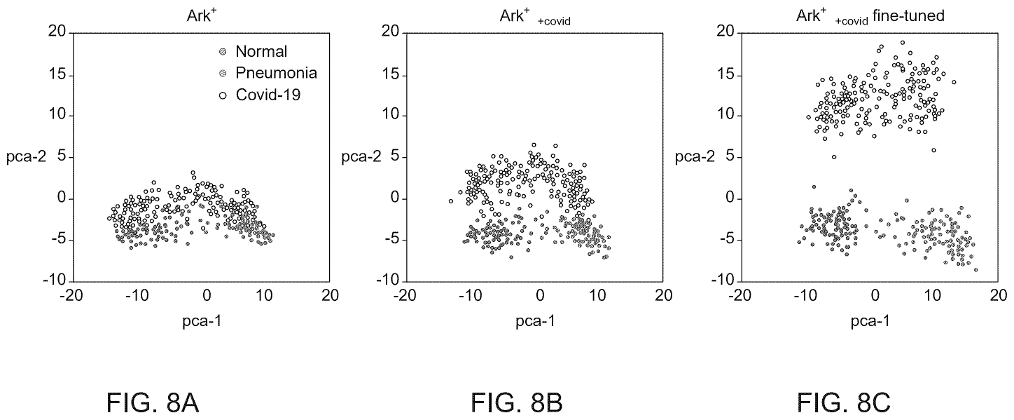

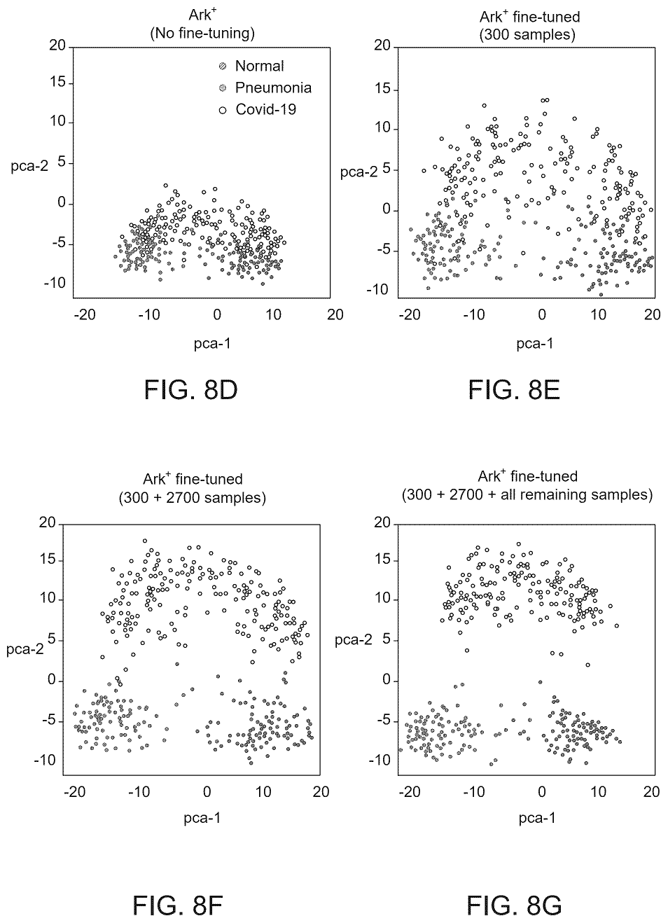

As shown in Table 5, Ark+, despite lacking prior exposure to COVID-19, consistently outperforms MIM-CXR, a self-supervised model pretrained with COVID-19 images, under both linear-probing and fine-tuning setups. The fine-tuned Ark+ model achieves superior accuracy of 98.83±0.26%, surpassing MIM-CXR, RAD-DINO, CXR-FM, and the state-of-the-art COVID-Net and Medical-MAE. Although Ark+'s linear-probing performance initially lags behind CXR-FM, incremental pretraining on the COVID-19 diagnostic task enables Ark+_{+covid} to surpass CXR-FM under identical configurations. To further illustrate the impact of the incremental pretraining, the t-SNE embeddings were visualized on the hold-out test data in COVID×CXR-3 generated by Ark+ and Ark+_{+covid} (see FIGS. 8A-8C). FIGS. 8A-8B illustrate how the embeddings for COVID-19, Pneumonia and Normal evolve in t-SNE, according to the disclosed embodiments. From Ark+ to Ark++covid, an upgraded Ark+ model is created by incrementally and continually pretraining Ark+ with the COVID-19 diagnostic task. Ark+_{+covid} has more distinct embeddings for the three conditions, revealing its newly acquired capacity for capturing the features specific to COVID-19. This capability can be further enhanced through fine-tuning as illustrated in FIG. 8C. The embeddings of COVID-19 cases show further segregation from pneumonia and normal cases after pretraining, highlighting the model's discriminative prowess to capture COVID-19-specific features and improve diagnostic accuracy. Furthermore, FIGS. 8D-8G demonstrate the evolution of t-SNE embeddings as Ark+ is fine-tuned with increasing numbers of samples, showcasing its efficient adaptation to the novel COVID-19 condition. These figures illustrate how the embeddings for COVID-19, Pneumonia and Normal evolve in t-SNE from the pretrained Ark+ to fine-tuning Ark+ with increasing numbers of samples continually. Ark+ obtains distinguishable embeddings when the training data reach 3,000, representing 10% of the full training set. This highlights Ark+'s ability to efficiently develop distinct feature representations, significantly enhancing its diagnostic accuracy and adaptability to new information. These results emphasize Ark+'s adaptability and extensibility for addressing previous unseen diseases during pretraining or potentially novel conditions, such as future pandemics. By combining incremental learning with high diagnostic performance, Ark+ demonstrates its value as a reliable tool for rapidly evolving clinical needs.

Table 5 shows the results of evaluating Ark+'s transferability to a novel disease. Ark+'s transferability to a novel disease, COVID-19, which Ark+ has never encountered in its pretraining, was evaluated. Via linear-probing, Ark+ performed significantly better than MIM-CXR (p-value: 1.98E-04), on a par with RAD-DINO (p-value: 1.38E-01), and significantly inferior to CXR-FM (p-value: 3.44E-06) in Accuracy (ACC), while Ark+ outperforms MIM-CXR and RAD-DINO via fine-tuning and overtakes CXR-FM's linear-probing performance. However, once Ark+ has been incrementally and continually pretrained with the COVID-19 diagnostic task, the resultant upgraded model, named Ark+_{+covid}, surpasses CXR-FM via linear-probing. The findings underscore the importance of openness in foundation models for continual pretraining, which endows Ark+ with the adaptability and scalability necessary to address novel diseases and potential future pandemics. Medical-MAE achieved an accuracy of 97.3% and COVIDNet holds an official accuracy score of 98.3% with their best-performing models.

TABLE 5
Performance comparison on COVID-19 diagnostic task

| Model | ACC(%) | meanAUC(%) | meanMCC(%) | meanAP(%) | meanF1(%) |
|---|---|---|---|---|---|
| fine-tuning* | | | | | |
| MIM-CXR | 95.88 ± 1.06 | 99.11 ± 0.47 | 93.88 ± 2.02 | 97.10 ± 1.65 | 95.67 ± 1.37 |
| RAD-DINO | 97.05 ± 0.27 | 99.64 ± 0.14 | 95.58 ± 0.81 | 99.08 ± 0.30 | 96.81 ± 0.56 |
| Ark⁺ | 98.83 ± 0.26 | 99.87 ± 0.14 | 97.35 ± 0.32 | 99.60 ± 0.19 | 98.59 ± 0.47 |
| Ark⁺_{+covid} | 99.08 ± 0.26 | 99.96 ± 0.01 | 97.72 ± 0.17 | 99.89 ± 0.03 | 98.33 ± 0.03 |
| Linear-probing | | | | | |
| CXR-FM | 98.60 ± 0.46 | 99.80 ± 0.04 | 97.07 ± 0.32 | 99.45 ± 0.09 | 98.14 ± 0.27 |
| MIM-CXR | 95.50 ± 0.16 | 99.16 ± 0.14 | 91.87 ± 0.49 | 97.60 ± 0.26 | 94.57 ± 0.38 |
| RAD-DINO | 95.85 ± 0.14 | 99.07 ± 0.09 | 92.28 ± 0.33 | 98.08 ± 0.16 | 94.58 ± 0.25 |
| Ark⁺ | 95.87 ± 0.20 | 99.09 ± 0.10 | 93.04 ± 0.28 | 97.80 ± 0.33 | 95.49 ± 0.19 |
| Ark⁺_{+covid} | 99.10 ± 0.12 | 99.90 ± 0.06 | 97.92 ± 0.21 | 99.61 ± 0.36 | 98.97 ± 0.16 |

### 2.8 Protect Privacy & Distribute Pretraining

Ark+ adopts centralized pretraining by default due to the absence of privacy concerns with public datasets. However, the concept underlying Ark+ is not restricted to public data, particular modality, or specific architecture. Given its neutrality in architecture and modality, Ark+ can utilize various backbones to accommodate different modalities (e.g., CT, MRI, videos, audios, tables, and text) and multimodal clinical data. Developing large multimodal AI models for medicine with large clinical data requires safeguarding patient privacy and distributing pretraining across multiple sites. To this end, Ark+ may be extended to federated Ark+ to support federated learning by deploying a (local) instance of Ark+ at each site as illustrated in FIG. 7. Ark+ can be federated by deploying a (local) Ark+ at each site to protect privacy and distribute training. In this setup, each local site trains its own Ark+ with all its available data, employing the same cyclic training strategy to train the student and the same epoch-wise EMA to update the teacher. After completing a round of local training, all sites send their student weights to a central server, where weights are averaged to aggregate these local models into a “master” model, consolidating knowledge from all sites. This master model is then distributed back to the local sites, allowing iterative learning and continuous improvement of the local teacher model. For simplicity, the projectors and multi-task heads are omitted from the illustration. Each local Ark+ is trained independently on the datasets available at its respective site. The model weights from these local instances are then collected by a central server, aggregated using a weight averaging operation, and redistributed back to the local sites to continue training collaboratively. A federated learning scenario was simulated by distributing one, two, and three pretraining datasets across three local sites, each hosting an instance of Ark+. Although this simulation used only public datasets, the datasets at each local site may be considered “private” and “inaccessible” by other sites. The experimental results, presented in Table 6, demonstrate the effectiveness of federated Ark+ in handling heterogeneous annotations across private clients, a capability novel to conventional federated learning, as well as the performance gains from federating data compared with isolated (local site) training, empowering researchers around the globe to confidently federate private data while ensuring patient privacy and advancing large open foundation models for medicine. Federated Ark+ is discussed further in Section 5 below.

With reference to Table 6, training federated Ark+ with data distributed across multiple clients maintains high performance on each task, underscoring the effectiveness and viability of distributed training for Ark+. The performance of federated Ark+ was evaluated for each task using their corresponding local teacher models, with predictions generated from the multi-task heads immediately after distributed training, without any additional fine-tuning. For a comprehensive comparison, isolated (local site) training was included, where a local Ark+ is trained with all datasets available at a particular site but no datasets are federated across sites. For Sites #2 and #3, federated training achieves superior performance compared with isolated (local site) training, highlighting the advantages of federated training and performance gains from federating privacy-preserving data over isolated (local site) training. While federated Ark+ can offer competitive performance in scenarios requiring privacy protection and distributed computing across multiple sites, centralized training is preferred when feasible, as it eliminates the communication overhead and allows the model to achieve superior overall performance

TABLE 6
Performance comparison among centralized, isolated (local site), and federated Ark+

| Pretraining task | 3-site | Isolated (local site) training | Federated training | Centralized training |
|---|---|---|---|---|
| MIMIC-II | Site #1 | 79.67 | 79.41 | 80.37 |
| CheXpert | Site #2 | 87.60 | 88.83 | 89.60 |
| RSNA Pneumonia | | 75.45 | 75.78 | 75.56 |
| ChestX-ray14 | Site #3 | 81.61 | 83.16 | 84.15 |
| VinDr-CXR | | 94.63 | 96.17 | 96.63 |
| Shenzhen | | 98.66 | 99.29 | 99.29 |
| Average | | 86.27 | 87.06 | 87.60 |

### 3 Discussion

The disclosed embodiments are referred to herein as Ark+, an open foundation model applied to chest radiography and pretrained cyclically by accruing and reusing the knowledge embedded in heterogeneous expert labels from six public datasets. Through eight clinical scenarios, Ark+'s generalizability, adaptability, robustness, extensibility, and superior performance is demonstrated over nine other foundation models in diagnosing thoracic diseases. Ark+ is generalizable. It surpasses other large-scale pretrained models on the test data of ChestX-ray14, underlining its generalizability in diagnosing common thoracic diseases (FIG. 2A). Supervised models Ark+ and CXR-FM consistently outperform self-supervised models MIM-CXR, CheSS, and RAD-DINO, indicating the power of expert knowledge. Fine-tuning further amplifies Ark+'s advantage over CXR-FM, signifying the foundation model's openness. Moreover, Ark+ generates more comprehensive diagnostic predictions through its multi-task heads, a capability that expands the diagnostic scope and addresses potential misdiagnosis (FIGS. 2B-2C).

Ark+ is adaptable. Despite being pretrained on only global labels of VinDr-CXR, it adapts to diagnostic tasks expanded with the lesion-level labels (FIGS. 3A-3C). This adaptability obviates the requirement for extensive retraining for evolving diagnostic needs. Ark+ also surpasses CXR-FM in detecting rare conditions with only 1-5 samples (FIG. 3D), emphasizing its clinical utility in data-scarce environments. Most importantly, Ark+ accommodates diagnostic setting shifts, achieving high AUC scores in detecting four diseases with unseen datasets via zero-shot transfer (FIGS. 5A-5D). The ability to transfer to new sites without training highlights its potential for real-world deployment.

Ark+ is robust. It surpasses other foundation models on the unseen ChestDR dataset when handling long-tailed disease distributions (FIGS. 4A-4C). In limited-data scenarios, linear probing outperforms fine-tuning, underscoring the importance of large-scale pretraining that yields strong features, overcoming overfitting and making simpler models more effective. With sufficient data, fine-tuning exceeds linear probing, showcasing an additional advantage of open and adaptable models. Furthermore, Ark+ naturally tolerates sex-biased data without specific designs for bias mitigation (Table 4). This capability advances equitable and accurate diagnostics across demographic groups, addressing ethical concerns in clinical AI. Future model bias evaluations should extend to attributes such as race, age, and other demographic factors to ensure more comprehensive and inclusive assessments of robustness.

Ark+ is extensible. By incorporating a (new) COVID-19 diagnostic task, Ark+ responded effectively to COVID-19 diagnosis (Table 5) via its incremental learning capability, highlighting its potential to extend to emerging diseases and future pandemics. Through its extension to federated learning (FIGS. 2A-2C), federated Ark+ preserves patient privacy and distributes pretraining across clients. This novel capability overcomes a limitation with conventional federated learning when handling heterogeneous annotations across clients, empowers privacy-preserving data sharing for developing large public foundation models, and positions Ark+ as a robust, scalable, and privacy-aware framework for open medical AI.

Ark+ is open, public, light, and affordable. Ark+ is relatively small, making its training affordable and efficient. All training data and labels are sourced from public datasets, and Ark+ helps eliminate the need for manual consolidation of heterogeneous labels, reducing data costs to near zero. Its training takes approximately 700 hours using 4 A100 GPUs. Ark+'s openness not only allows fine-tuning and incremental learning with new data to enhance diagnostic accuracy in various scenarios (FIGS. 2A-2C and 4A-4C, Table 5), but also enables its extension from classification to localization, segmentation, and their integration. Ark+ is pretrained with data sourced predominantly from populations in the USA, Vietnam, and China, but it is hoped that its public nature will attract more diverse data sources, especially from underrepresented regions and demographic groups, to better its generalizability. Its lightness and affordability will encourage quick replication, public evaluation, and local adoption, and anticipating its full openness will foster future collaborative developments of Ark+ from AI specialists for particular organs, specialties, and modalities (e.g., heart, fundus; pathology, dermatology; CT, MRI) to AI generalists for medicine trained with multimodal clinical data (e.g., text, tables, images, videos, and audios).

In summary, Ark+ demonstrates impressive performance in diagnosing thoracic diseases across various scenarios. Its generalizability, adaptability, robustness, extensibility, openness, lightness, and affordability make it a powerful model fundamental to medical imaging for the public. Its exceptional capabilities are attributable to a simple yet powerful insight: aggregating numerous (public or private) datasets (large or small) and utilizing their available annotations incurs minimal costs while substantially increasing data size, expanding protocol coverage, diversifying patient populations, and accruing expert knowledge from a broad spectrum of global sources. Ark+ attests that accruing and reusing knowledge from heterogeneous expert annotations with even only public datasets can surpass the performance of proprietary models trained on unusually large data. Considering the ubiquity of heterogeneous data and labels across various fields, including biology, chemistry, physics, and medicine, thanks to its neutrality in modality and architecture, the concept underlying Ark+ is expected to have far-reaching potential beyond imaging.

### 4 Method

### 4.1 Pretraining Ark+ Using Numerous Public Datasets

Ark+ learns superior and robust visual representations from large-scale aggregated medical images by accruing and reusing the expert knowledge embedded in all available heterogeneous labels. FIG. 6 depicts Ark+'s pretraining framework, which is a teacher model and a student model, each augmented with multi-task heads (each corresponding to one task) and trained via cyclic pretraining. Cyclic pretraining is an iterative process: at each iteration, the student accrues knowledge from every expert annotation through its corresponding task head by sequentially scanning all datasets (tasks) individually and sequentially for one epoch. At the end of each task, the knowledge accrued by the student is accumulated into the teacher via exponential moving averages (EMA) and reused to facilitate the student accruing more knowledge from the expert annotations associated with the next dataset (task). To reinforce the feedback loop between the student and teacher, a projector is introduced after their encoders to map the representations to the same feature space via the consistency loss, thereby also serving as the embedding for linear-probing in the evaluation. After pretraining, the teacher's accumulated knowledge can be reused and transferred to target tasks. Differing from the previous design in Ark, Ark+ feeds the teacher model with the resized original image rather than using random cropping. This data augmentation update ensures the teacher provides a consistent and steady supervisory signal for computing the consistency loss, thereby accelerating training and enhancing performance. To emphasize the strengths of Ark+'s pretraining framework, ablation studies on the multi-task head design, the cyclic training approach, and the performance improvements achieved with Ark+ are included in the Supplementary Methods (Sec. 6).

Pretraining setup. Ark+ was pretrained with 704,363 chest radiographs from six datasets (detailed in Table 2) collected from six different institutions around the world and annotated by their experts. The labels originally provided were utilized without manually consolidating the heterogeneous labels into a pre-defined list. To avoid test-image leaks, all validation and test data were excluded from the Ark+ pretraining. Ark+ leverages the large version of the Swin transformer with an input resolution of 768×768 as the backbone. The teacher and student encoders were initialized with the officially released weights trained on ImageNet (GitHub.com/SwinTransformer/storage/releases/download/v1. 0.0/swin_large_patch4_window12_384_22ktolk.pth), and the projectors and the multi-task heads were randomly initialized. The student model was trained with both classification and consistency loss. The classification loss (L_{cls}) was tailored to each dataset's labels, using binary cross-entropy for binary/multi-label tasks and cross-entropy for multi-class tasks. The consistency loss (L_{consist}) was optimized using mean-squared error. Training employed an SGD optimizer with an initial learning rate of 0.3, a batch size of 50 across 4 Nvidia A100 GPUs with 80 GB memory each. The teacher model was updated using an epoch-wise EMA based on the student's one epoch of learning at the end of each task, using a momentum of 0.9. Image augmentations include random cropping and rotation, as well as changes in brightness, contrast, and Gamma distribution. The model was pretrained for 50 epochs, iterating through all datasets 50 times. After pretraining, the teacher model, including the encoder, projector, and multi-task heads, was deployed for the clinical target tasks via fine-tuning, linear-probing, and zero-shot transfer.

### 4.2 Datasets for Developing and Evaluating Ark+

Pretraining datasets. Table 7 summarizes the six datasets used for pretraining Ark+ in which Ark+ is pretrained on 704,363CXR images sourced from six public datasets collected from six different institutions worldwide, using readily accessible but heterogeneous expert labels. MIMIC-II (i.e., MIMIC-CXR v2.0) contains 377,110 frontal and lateral chest radiographs and 14 structured labels derived from the 227,827 free-text radiology reports using CheXpert. The radiographic studies were collected at Beth Israel Deaconess Medical Center in Boston, MA between 2011 and 2016. CheXpert consists of 224,316 CXRs of 65,240 patients collected at Stanford Hospital between October 2002 and July 2017, including both frontal and lateral views. Each study was labeled for the presence of 14 observations as positive, negative, or uncertain. The NIH ChestX-ray14 dataset comprises 112,120 frontal-view CXRs collected from 30,805 unique patients, obtained from radiological reports spanning the years 1992 to 2015. Each image is associated with 14 common disease labels overlapping with CheXpert but not identical. The RSNA Pneumonia Detection Challenge (RSNA Pneumonia), created in collaboration with the Radiological Society of North America (RSNA), comprises 30,000 frontal-view CXRs sourced from the NIH. These images are annotated with image-level labels indicating the presence of lung opacity and any abnormalities. VinDr-CXR contains 18,000 postero-anterior (PA) view CXRs annotated with the localization of 22 critical findings (local labels) and six thoracic diagnoses (global labels). The images were collected from two of Vietnam's largest hospitals between 2018 and 2020. Shenzhen Hospital X-ray Set (Shenzhen) comprises 662 frontal-view CXRs, including 326 normal X-rays and 336 abnormal X-rays showing various manifestations of tuberculosis. The images were collected by Shenzhen No. 3 Hospital in Shenzhen, Guangdong Province, China in 2012. If the dataset provides an official data split, the training set data was used for pretraining; otherwise, 70% of the data was randomly allocated for pretraining purposes.

TABLE 7
Overview of datasets utilized for pretraining Ark+

| Datasets | Classification Task | Country | Collection Institute | #Pretrain |
|---|---|---|---|---|
| MIMIC-II | Multi-label | USA | BIDMC | 368,879 |
| CheXpert | Multi-label | USA | Stanford Hospital | 223,414 |
| ChestX-ray14 | Multi-label | USA | NIH | 75,312 |
| RSNA Pneumonia | Multi-class | USA | RSNA | 21,295 |
| VinDr-CXR | Multi-label | Vietnam | VinBigData | 15,000 |
| Shenzhen | Binary | China | Shenzhen No. 3 Hospital | 463 |

Data with rare diseases. CXR-LT expands upon MIMIC-II by increasing the target classes from 14 to 26, incorporating labels for 12 additional disease findings derived from parsing radiology reports. Three rare diseases from the newly added labels were used for the few-shot linear-probing benchmark.

Data in long-tailed distribution. ChestDr offers 4,848 frontal chest radiography images (from 4,848 patients) gathered from two regional hospitals in Hubei and Jiangxi Province, China. This dataset's initial disease labels were assigned by a radiological resident, supported by previously signed radiology reports, and subsequently verified by a senior radiologist. The dataset exhibits a long-tailed distribution, comprising 10 head classes with sample numbers ranging from 429 to 1300 and nine tail classes with sample numbers ranging from 23 to 305.

Data for zero-shot transfer. SIIM-ACR, initially introduced for pneumothorax segmentation, includes a total of 12,047 CXRs with 2,669 cases of Pneumothorax. TBX11K released 8,400 CXRs from the official training and validation sets, including 3,800 healthy cases, 3,800 sick but non-tuberculosis cases, and 800 cases with manifestations of tuberculosis. Mendeley-V2 is a pediatric CXR dataset that includes 4,273 pneumonia images and 1,583 normal images. NODE21 represents a nodule detection challenge with 4,882 frontal CXRs, of which 1,134 CXRs are annotated with bounding boxes around nodules, while the remaining 3,748 images represent the negative class without nodules.

Data for COVID-19. COVID×CXR3 provides a dataset featuring over 30,000 C×R images sourced from a diverse multinational cohort of more than 16,400 patients, containing 16,490 positive COVID-19 images derived from a cohort of over 2,800 patients. Following Wang et al. Wang, L., Lin, Z. Q. & Wong, A., Covid-net: A tailored deep convolutional neural network designed for detection of covid-19 cases from chest x-ray images. Scientific Reports 10, 19549 (2020), and Xiao et al., Xiao, J., Bai, Y., Yuille, A. & Zhou, Z. Delving into masked autoencoders for multi-label thorax disease classification. In Proceedings of the IEEE/CVF Winter Conference on Applications of Computer Vision, 3588-3600 (2023), the label version V9A was utilized, which was curated for the detection and differentiation of patients without pneumonia, and patients with non-COVID-19 pneumonia, and COVID-19 pneumonia.

### 4.3 Large-Scale Pretrained Foundation Models for Comparison

CXR-FM is a proprietary CXR Foundation Model (CXR-FM) pretrained on 821,544 labeled and mostly private CXRs from three different sources using supervised contrastive learning (SupCon).

ELIXR is an advanced extension of CXR-FM, integrating a language-aligned image encoder with a fixed large language model. It is pretrained on 650,264 private CXRs and 243,324 images from the MIMIC-CXR dataset, along with their associated free-text radiology reports.

RAD-DINO is a publicly accessible vision transformer model, pretrained on 882,775 CXRs from five public, deidentified datasets using the state-of-the-art self-supervised learning approach, i.e, DINOv2.

MIM-CXR is a publicly accessible self-supervised model pretrained on 926,028 CXRs from 13 public, deidentified datasets using masked image modeling, i.e., SimMIM.

CheSS is a publicly accessible pretrained model trained with a 4.8 million in-house CXR dataset using self-supervised contrastive learning, i.e., MoCo v2.

KAD refers to an image-text model pretrained using the Knowledge-enhanced Auto Diagnosis approach, which leverages a medical knowledge base to guide vision-language pretraining with paired chest radiographs and radiology reports from MIMIC-CXR.

XRV ResNet-50 and DenseNet-121 are two official models from torchxrayvision (XRV) pretrained using over 200,000 unique chest radiographs from four public datasets, each filtered to include only one AP or PA view per patient.

### 4.4 Evaluation Metrics and Statistical Analysis

To offer a comprehensive assessment of a model's classification performance, the following metrics were adopted, focusing on different aspects, such as discrimination ability, overall accuracy, balance between precision and recall, and handling of class imbalances:

Area Under the ROC Curve (AUC) evaluates the performance on binary classification tasks and represents the area under the Receiver Operating Characteristic (ROC) curve, which plots the true positive rate (sensitivity) against the false positive rate (1-specificity) at various threshold values. A higher AUC value (closer to 1) indicates better positive and negative class model discriminatory ability.

Accuracy (ACC) measures the proportion of correctly classified instances (both true positives and true negatives) relative to the total number of instances, commonly used for evaluating classification models, and is calculated as the ratio of correct predictions to total predictions.

The F1 score (F1) is the harmonic mean of both precision and recall, providing a balanced assessment of a classification model's performance, especially in cases of class imbalance.

Average Precision (AP) evaluates the performance of models in precision-recall curves, particularly for binary classification tasks. This measure calculates the area under the precision-recall curve, providing an indication of how well the model balances precision and recall across different thresholds.

Matthews Correlation Coefficient (MCC) measures the quality of binary classifications, considering true positives, true negatives, false positives, and false negatives, ranging from −1 to 1:1 indicates perfect prediction, 0 indicates random prediction, and −1 indicates total disagreement between predictions and actual labels. MCC is particularly useful for imbalanced datasets as it considers all four elements of the confusion matrix.

The threshold for computing ACC, F1, and MCC is determined according to the Youden Index, which involves selecting a threshold that maximizes the difference between the true positive rate and the false positive rate. In other words, the threshold that optimally balances the ability to detect true positives while minimizing false positives was determined.

Statistical analysis incorporating independent two-sample t-test and 95% confidence intervals is utilized to determine the significance among model performances and draw reliable conclusions. In experiments involving fine-tuning and linear-probing, as the linear classifier is randomly initialized, each run produces varying results. Hence, evaluations for each model were conducted at least 10 times, reporting performance metrics mean and standard deviation and performing statistical analysis using the two-sided independent t-test. For experiments utilizing zero-shot transfer, the model produces consistent results as all components remain frozen. To obtain reliable estimates, 95% confidence intervals were derived through bootstrapping techniques on the test set, creating 1,000 resampled datasets. Within each resampled dataset, the AUC score was computed and a confidence interval was constructed to encapsulate its variability. This iterative approach, repeated 1,000 times, yields a distribution of statistics and corresponding confidence intervals, offering valuable insights into the variability and uncertainty inherent in the estimates.

### 5 Federated Ark+

Federated Learning (FL) enables collaborative model training across decentralized data sources while preserving data privacy. However, most existing FL methods assume label homogeneity across clients, which is rarely the case in real-world medical settings where datasets are annotated inconsistently. To tackle this challenge, Federated Ark+, an FL framework handles heterogeneous labels across clients. Federated Ark+ leverages multi-task heads and a cyclic training strategy, deploying local Ark+ models at clients and aggregating encoder weights at a central server for iterative refinement. Evaluation using six datasets across two federated scenarios demonstrates that Federated Ark+ achieves strong overall performance and significantly boosts outcomes for clients with limited data.

### 5.1 Introduction

Federated Learning (FL) is a collaborative learning paradigm that trains deep learning models across decentralized data sources while ensuring data privacy by keeping the raw data local. However, most current FL methods assume homogeneous data and identical labels among local clients. In practice, datasets collected from different institutions are often annotated inconsistently, leading to variations in disease label coverage across participating clients (e.g., hospitals and medical centers). Some approaches circumvent this problem using self-supervised learning methods, but they discard the valuable supervision provided by expert labels. Therefore, this discussion aims to address a critical question: How can label heterogeneity be effectively handled in supervised FL? To answer the question, the disclosed embodiments propose Federated Ark+, a framework designed to handle heterogeneous labels across datasets and clients in FL settings for fully-supervised learning. Federated Ark+ is an extension and application of Foundation Ark+ to FL, leveraging Ark+'s multi-task heads and cyclic training strategy to address label heterogeneity across datasets. It deploys local Ark+ instances at clients, aggregates the encoder model weights at the central server, and redistributes the aggregated model for continued local training. Experimental results under two federated scenarios demonstrate that Federated Ark+ maintains strong overall performance and significantly improves outcomes for clients with limited data.

### 5.2 Federated Ark+

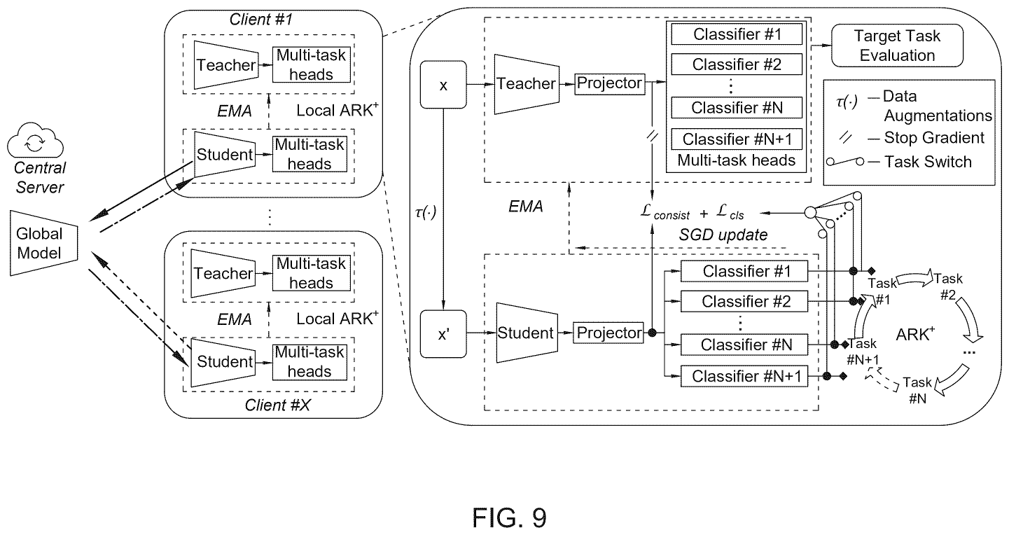

Local Training: Ark+ is a framework to pretrain foundation models by Accruing and Reusing Knowledge from heterogeneous expert labels in various datasets (see FIG. 9). Federated Ark+ aims to address label heterogeneity in FL by deploying a local Ark+ instance at each client. Clients operate on their own data locally, preserving privacy, and pretrain their Ark+ models using a student-teacher framework, multi-task heads, and a cyclic training strategy. After each training round, student model weights are sent to a central server for aggregation into a global model, which is then redistributed for continued local training. This iterative process enhances the local teacher models, particularly benefiting tasks with limited data. It is built on a teacher-student model, augmented with multi-task heads (each corresponding to one task), and trained cyclically. Cyclic training is an iterative process where the student learns from expert annotations via classification loss (L_{cls}), sequentially scanning each dataset through its corresponding task head. After each task (one epoch), the student's knowledge is integrated into the teacher using exponential moving averages (EMA). A projector aligns student and teacher representations through a consistency loss (L_{consist}), reinforcing their interaction. After pretraining, the accrued knowledge in the teacher can be reused and transferred to target tasks.

Federated Aggregation: During the training of the local Ark+ at each client, once all clients have completed a round of local training, the weights of their student models are transmitted to a central server for aggregation and redistribution. To keep the process efficient and scalable, the central server employs a straightforward weight averaging strategy to merge these local models into a global model. This approach allows the system to synthesize diverse knowledge learned across clients, even when each client operates on a distinct set of labels.

Tackling Label Heterogeneity: Ark+'s multi-task heads play a key role in handling label heterogeneity locally. Each head is tailored to a specific task or label set, enabling the client to learn effectively from its own data distribution. If necessary, the central server can also aggregate the weights of these multi-task heads, constructing a global model capable of addressing label heterogeneity across the entire federated network. This flexibility ensures that Federated Ark+ not only preserves data privacy and autonomy at each site but also maintains adaptability in handling diverse and unevenly distributed label spaces.

### 5.3 Experiments and Results

Datasets: Federated Ark+ was evaluated on six public chest radiography datasets with heterogeneous labels: MIMIC-CXR (377K images), CheXpert (224K), RSNA Pneumonia (27K), ChestX-ray14 (112K), VinDr-CXR (18K), and Shenzhen-CXR (662). Each dataset is labeled for varying thoracic conditions. The official splits and Ark+ protocol were followed for training and evaluation.

FL Settings: The six datasets were distributed across clients and two scenarios evaluated: (1) 6-client—each client receives one dataset; (2) 3-client—each client receives one, two, or three datasets, respectively (see Table 8). Each client trains a local Ark+ model on its own data and shares student weights with a central server for aggregation. Teacher models are updated via EMA, following the centralized Ark+ setup. 50 communication rounds are performed. For comparison, isolated training is included, where a local Ark+ is trained on all datasets at a single site without federation.

TABLE 8

| Task | Label No. | | Metrics | Centralized | 6-client | Isolated | Federated | 3-client | Isolated | Federated |
|---|---|---|---|---|---|---|---|---|---|---|
| MIMIC-CXR | 14 | labels | AUC | 80.37 | Client #1 | 79.67 | 78.68 | Client #1 | 79.67 | 79.41 |
| CheXpert | 14 | labels | AUC | 89.60 | Client #2 | 88.08 | 87.93 | Client #2 | 87.60 | 88.33 |
| RSNA Pneu. | 3 | classes | Accuracy | 75.56 | Client #3 | 74.81 | 75.38 | | 75.45 | 75.78 |
| Chest X-ray14 | 14 | diseases | AUC | 84.15 | Client #4 | 82.98 | 82.54 | Client #3 | 81.61 | 83.16 |
| VinDr-CXR | 6 | conditions | AUC | 96.63 | | 91.41 | 95.44 | | 94.63 | 96.17 |
| Shenzhen-CXR | 2 | classes | AUC | 99.29 | Client #6 | 96.43 | 99.02 | | 98.66 | 99.29 |

| | | | | | |
|---|---|---|---|---|---|
| Average performance (%) | 87.60 | 85.56 | 86.50 | 86.27 | 87.06 |

Results and Analysis: Table 8 compares centralized training, isolated (local) training, and Federated Ark+ under 3-client and 6-client configurations. Compared with centralized and isolated training, Federated Ark+ achieves consistently high performance across tasks, demonstrating its effectiveness in federated learning settings. Performance is evaluated per task using local teacher models, with predictions from multi-task heads obtained directly after training. Federated Ark+ achieves comparable overall AUCs of 87.06% (3-client) and 86.50% (6-client) versus 87.60% from centralized training. It notably boosts performance for clients with limited data (e.g., VinDr-CXR and Shenzhen-CXR) over isolated training. Slight drops are observed for clients with large datasets (e.g., MIMIC-CXR, CheXpert, ChestX-ray14), likely due to naive weight averaging during aggregation, which may underrepresent larger datasets. This could be addressed with more adaptive strategies, such as data-size-aware weighted averaging.

### 5.4 Summary

Federated Ark+ is a framework for addressing heterogeneous labels across datasets and clients in FL. Results show that it maintains strong overall performance while notably improving outcomes for clients with limited data.

### 6 Supplementary Methods

### Ablation Study

To showcase the advantages of the Ark+ framework, ablation studies were conducted that highlight its multi-task head design (Sec. 6.1) and cyclic training approach (Sec. 6.2). Additionally, to further illustrate the performance improvements achieved with Ark+, fine-tuning baselines were provided, initialized from an ImageNet-pretrained model for comparison (Sec. 6.3).

6.1 Multi-Task Heads Vs. Single-Task Head

Compared with a single-task head, the multi-task head design of Ark+ delivers superior performance (see Table 10) and provides several key benefits:
1. Reduce manual effort: The multi-task heads design eliminates the need for prior label consolidation: “understanding” and pooling all labels from different datasets into a predefined list (Table 9), simplifying the process.
2. Increase flexibility and scalability: Pluggable heads offer easy integration of new tasks without requiring retraining or extending the head's dimension.
3. Lower code complexity: Task-specific heads avoid the complexity of managing class indices for loss computation, unlike the single-task head, which must map predictions to specific datasets' ground truth.

TABLE 9
Manually assembling the labels from five datasets into a predefined list

| | Task 1 | Task 2 | Task 3 | Task 4 | Task 5 |
|---|---|---|---|---|---|
| Index | [CheXpert] | [ChestX-ray14] | [RSNA Penumonia] | [VinDr-CXR] | [Shenzhen] |
| 0 | No Finding | | Normal | No finding | |
| 1 | Enlarged | | | | |
| | Cardiomediastinum | | | | |
| 2 | Cardiomegaly | Cardiomegaly | | | |
| 3 | Lung Opacity | | Lung Opacity | | |
| 4 | Lung Lesion | | | | |
| 5 | Edema | Edema | | | |
| 6 | Consolidation | Consolidation | | | |
| 7 | Pneumonia | Pneumonia | | Pneumonia | |
| 8 | Atelectasis | Atelectasis | | | |
| 9 | Pneumothorax | Pneumothorax | | | |
| 10 | Pleural Effusion | Effusion | | Pleural Effusion | |
| 11 | Pleural Other | | | | |
| 12 | Fracture | | | | |
| 13 | Support Devices | | | | |
| 14 | | Infiltration | | | |
| 15 | | Mass | | | |
| 16 | | Nodule | | | |
| 17 | | Emphysema | | | |
| 18 | | Fibrosis | | | |
| 19 | | Pleural_Thickening | | | |
| 20 | | Hernia | | | |
| 21 | | | No Lung Opacity/ | | |
| | | | Not Normaal | | |
| 22 | | | | Lung tumor | |
| 23 | | | | Tuberculosis | Tuberculosis |
| 24 | | | | Other diseases | |

TABLE 10
Ablation study comparing the multi-task heads design with the single-task head used in Ark+

| | | Task 1 | Task 2 | Task 3 | Task 4. | Task 5 |
|---|---|---|---|---|---|---|
| Test using the pretrained head(s) | | [CheXpert] | [ChestX-ray14] | [RSNA Penn.] | [VinDr-CXR] | [Shenzhen] |
| Pretraining tasks | Design | mAUC (%) | mAUC (%) | ACC (%) | mAUC (%) | AUC (%) |
| Task 2, 4 | Multi-task heads | — | 79.89 | — | 94.14 | — |
| | Single task head | — | 79.88 | — | 93.45 | — |
| Task 1, 2, 3, 4, 5 | Multitask heads | 88.12 | 81.38 | 74.05 | 95.5 | 99.6 |
| | Single-task head | 88.09 | 80.94 | 73.67 | 94.96 | 98.06 |

| | | |
|---|---|---|
| | | VinDr-CXR |
| Linear-probing | ChestDR | (all 28 classes) |

| Pretraining tasks | Design | mAUC (%) | mAUC (%) |
|---|---|---|---|
| Task 2, 4 | Multi-task heads | 80.75 ± 0.13 | 91.25 ± 0.09 |
| | Single-task head | 80.21 ± 0.13 | 90.92 ± 0.06 |
| Task 1, 2, 3, 4, 5 | Multi-task heads | 82.24 ± 0.12 | 93.28 ± 0.06 |
| | Single-task head | 82.42 ± 0.05 | 92.82 ± 0.07 |

As presented in Table 9, labels from different datasets were manually consolidated to train a single-task head for comparison with Ark+'s multi-task heads. All other experimental settings remained identical. To reduce training costs, we used the Swin-Base backbone with an input resolution of 224×224. Two sets of experiments were conducted using two and five pretraining tasks. After pretraining, the heads on hold-out test data of the pretraining tasks were evaluated through end-to-end inference and linear-probing conducted on the ChestDR and VinDr-CXR datasets to assess embedding quality. The results show that Ark+ with multi-task heads consistently outperforms the single-task head, except in the external evaluation on ChestDR for models pretrained with five tasks.

6.2 Cyclic Training Vs. Simultaneous Training

Cyclic training enhances model performance more effectively than simultaneous training. Although all data is centralized and accessible, cyclic training was chosen over simultaneous training to enhance model performance across multiple tasks. Simultaneous training involves updating the model using a combined loss from all tasks, which can lead to conflicting gradients during back-propagation. This conflict may weaken the overall gradient signal, causing slower convergence and suboptimal performance. By contrast, cyclic training allows the model to focus on one task at a time in each iteration, reducing gradient interference and promoting more efficient learning. To demonstrate this, an ablation study was conducted comparing cyclic training with simultaneous training. In the simultaneous training scenario, mini-batches are constructed by randomly sampling from all datasets, and losses are calculated based on the respective dataset IDs and labels. As shown in Table 11, Ark+ with cyclic training demonstrates superior performance over simultaneous training. This confirms that cyclic training is a more effective approach in this context.

TABLE 11
Ablation study comparing cyclic training with simultaneous training using five pretraining tasks (as listed in Table 9)

| | | Task 1 | Task 2 | Task 3 | Task 4 | Task 5 |
|---|---|---|---|---|---|---|
| Test using the pretrained head(s) | | [CheXpert] | [ChestX-ray14] | [RSNA Penn.] | [VinDr-VXR] | [Shenzhen] |
| Pretraining tasks | Method | mAUC (%) | mAUC (%) | ACC (%) | mAUC (%) | AUC (%) |
| Task 1, 2, 3, 4, 5 | Cyclic training | 88.12% | 81.95% | 74.05% | 95.50% | 99.60% |
| | Simultaneous training | 87.02% | 80.93% | 71.61% | 94.37% | 98.62% |

| | | |
|---|---|---|
| | | VinDr-CXR |
| Linear-probing | ChestDR | (all 28 dasses) |

| Pretraining tasks | Method | mAUC (%) | mAUC (%) |
|---|---|---|---|
| Task 1, 2, 3, 4, 6 | Cyclic training | 82.24 ± 0.12 | 93.28 ± 0.08 |
| | Simultaneous training | 76.54 ± 0.72 | 91.47 ± 0.15 |

### 6.3 Performance Boosts Via Ark+ Pretraining

Ark+ is designed to train robust and high-performing foundation models by aggregating numerous datasets, both large and small, to learn from their heterogeneous annotations. As shown in Table 12, smaller datasets were included, such as VinDR-CXR and Shenzhen, to demonstrate that Ark+ can significantly enhance performance compared to fine-tuning each task individually, particularly for these smaller datasets. The results compare fine-tuning baselines initialized from an ImageNet-pretrained model against the Ark+ model, both using the Swin Transformer Large backbone and an input resolution of 768×768. Ark+ significantly boosts performance across tasks, particularly for smaller datasets such as VinDr-CXR and Shenzhen. As shown in Table 12, when fine-tuning the ImageNet-pretrained model on VinDR-CXR and Shenzhen, the performances were 91.41±0.96% and 96.43±0.81%, respectively. After pretraining with Ark+, the performance improved to 96.42±0.10% on VinDR-CXR and 99.07±0.06% on Shenzhen. These results validate the approach, demonstrating that pretraining on multiple datasets, including smaller datasets, substantially mitigates overfitting and improves generalizability, especially for bottleneck datasets.

TABLE 12
Ablation study demonstrating performance improvements
across six tasks achieved with Ark+

| Task | Metric | Fine-tuning baseline | Ark⁺ | Performance boost |
|---|---|---|---|---|
| MIMIC-II | mAUC(%) | 79.67 ± 0.28 | 80.67 ± 0.16 | +1.00 |
| CheXpert | mAUC(%) | 88.08 ± 0.14 | 89.67 ± 0.09 | +1.59 |
| ChestX-ray14 | mAUC(%) | 82.98 ± 0.16 | 84.43 ± 0.09 | +1.45 |
| RSNA Pneumonia | ACC(%) | 74.81 ± 0.40 | 75.73 ± 0.27 | +0.92 |
| VinDr-CXR | mAUC(%) | 91.41 ± 0.96 | 96.42 ± 0.10 | +5.01 |
| Shenzhen | AUC(%) | 96.43 ± 0.81 | 99.07 ± 0.06 | +2.64 |

Embodiments of the disclosure contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. The processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the disclosure provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk, read-only memories (ROMs), random access memories (RAMs), EPROMS, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. On the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the claims that follow, along with the full scope of equivalents to which such claims are entitled.

What is claimed is:

1. A method for interpreting medical images comprising:
cyclically pretraining an open foundation artificial intelligence (AI) model by accruing and reusing knowledge from heterogeneous expert labels embedded in a plurality of public datasets of medical images, the model comprising three pre-trained components: a pre-trained backbone encoder, a projector, and a plurality of multi-task heads, for use in clinical tasks via fine-tuning, linear-probing, and zero-shot transfer;
fine-tuning the model, via a randomly-initialized linear classifier coupled to the pretrained encoder, using the medical images and associated labels provided by a target task;
generating embeddings, via the pretrained backbone encoder and the projector, for all medical images in the target task;
training a new linear classifier; and
acquiring, via the pre-trained backbone encoder, projector, and the plurality of multi-task heads, a prediction directly for each medical image in the target task.

2. The method of claim 1, wherein fine-tuning the model, via the randomly-initialized linear classifier coupled to the pretrained encoder, using the medical images and associated labels provided by the target task, comprises pretraining a student-teacher network of the model, including a backbone encoder and the linear classifier.

3. The method of claim 2, wherein pretraining the linear classifier comprises training only the linear classifier atop frozen features extracted by the pretrained backbone encoder.

4. The method of claim 1, wherein acquiring, via the pre-trained backbone encoder, projector and the plurality of multi-task heads, the prediction directly for each medical image in the target task, comprises directly utilizing, via the zero-shot transfer, the model to diagnose conditions for datasets not seen during a pretraining phase thereby requiring no further training.

5. A pretraining framework for an AI model to learn visual representations from large-scale aggregated medical images by accruing and reusing the expert knowledge embedded in all available heterogeneous labels, the framework comprising:
a teacher model;
a student model;
wherein the teacher model and the student model are each augmented with multi-task heads, wherein each multi-task head corresponds to one task;
wherein the teacher model and student model are each trained via an iterative cyclic pretraining process, in which, at each iteration, the student model is to accrue knowledge from every expert annotation through its corresponding task head by sequentially scanning all tasks one by one for one epoch and, at the end of each task, the knowledge accrued by the student model is accumulated into the teacher model via exponential moving averages (EMA) and reused to help the student model accrue more knowledge from the expert annotations associated with a next task.

6. The pretraining framework of claim 5, further comprising a projector to map representations to a same feature space via a consistency loss and serve as an embedding for linear-probing in an evaluation to reinforce a feedback loop between the student model and the teacher model, after the encoders.

7. The pretraining framework of claim 5, wherein, after pretraining, the accumulated knowledge in the teacher is reused and transferred to target tasks.

8. The pretraining framework of claim 5, wherein the teacher model is fed with resized medical images to provide a consistent and steady supervisory signal for computing a consistency loss, thereby accelerating training and enhancing performance.

9. A system comprising:
a memory to store instructions;
a processor to execute the instructions stored in the memory to pretrain via a pretraining framework an AI model to learn visual representations from large-scale aggregated medical images by accruing and reusing the expert knowledge embedded in all available heterogeneous labels, the framework comprising:
a teacher model;
a student model;
wherein the teacher model and the student model are each augmented with multi-task heads, wherein each multi-task head corresponds to one task;
wherein the teacher model and student model are each trained via an iterative cyclic pretraining process, in which, at each iteration, the student model is to accrue knowledge from every expert annotation through its corresponding task head by sequentially scanning all tasks one by one for one epoch and, at the end of each task, the knowledge accrued by the student model is accumulated into the teacher model via exponential moving averages (EMA) and reused to help the student model accrue more knowledge from the expert annotations associated with a next task.

10. The system of claim 9, wherein the pretraining framework further comprises a projector to map representations to a same feature space via a consistency loss and serve as an embedding for linear-probing in an evaluation to reinforce a feedback loop between the student model and the teacher model, after the encoders.

11. The system of claim 9, wherein, after pretraining, the accumulated knowledge in the teacher is reused and transferred to target tasks.

12. The system of claim 9, wherein the teacher model is fed with resized medical images to provide a consistent and steady supervisory signal for computing a consistency loss, thereby accelerating training and enhancing performance.

13. A non-transitory computer-readable storage media having instructions stored thereupon that, when executed by a system having at least a processor and a memory therein, learn a foundation model for interpreting medical images, by executing the instructions via the processor for:
cyclically pretraining an open foundation artificial intelligence (AI) model by accruing and reusing knowledge from heterogeneous expert labels embedded in a plurality of public datasets of medical images, the model comprising three pre-trained components: a pre-trained backbone encoder, a projector, and a plurality of multi-task heads, for use in clinical tasks via fine-tuning, linear-probing, and zero-shot transfer;
fine-tuning the model, via a randomly-initialized linear classifier coupled to the pretrained encoder, using the medical images and associated labels provided by a target task;
generating embeddings, via the pretrained backbone encoder and the projector, for all medical images in the target task;
training a new linear classifier; and
acquiring, via the pre-trained backbone encoder, projector, and the plurality of multi-task heads, a prediction directly for each medical image in the target task.

14. The non-transitory computer-readable storage media of claim 13, wherein fine-tuning the model, via the randomly-initialized linear classifier coupled to the pretrained encoder, using the medical images and associated labels provided by the target task, comprises pretraining a student-teacher network of the model, including a backbone encoder and the linear classifier.

15. The non-transitory computer-readable storage media of claim 14, wherein pretraining the linear classifier comprises training only the linear classifier atop frozen features extracted by the pretrained backbone encoder.

16. The non-transitory computer-readable storage media of claim 15, wherein acquiring, via the pre-trained backbone encoder, projector and the plurality of multi-task heads, the prediction directly for each medical image in the target task, comprises directly utilizing, via the zero-shot transfer, the model to diagnose conditions for datasets not seen during a pretraining phase thereby requiring no further training.
